(19) United States
(12) Patent Application Publication
(10) Pub. No.: US 2026/0045342 A1
(43) Pub. Date: Feb. 12, 2026

(54) METHODS AND SYSTEMS FOR GENERATING RECOMMENDATIONS FOR HEALTH AND WELLNESS

(71) Applicant: Docfully, Inc., San Diego, CA (US)

(72) Inventor: Jacques STROUD, San Diego, CA (US)

(21) Appl. No.: 19/296,637

(22) Filed: Aug. 11, 2025

Related U.S. Application Data
(60) Provisional application No. 63/682,184, filed on Aug. 12, 2024.

(51) Int. Cl. G16H 20/00; G16H 10/60; G16H 15/00; G16H 50/20; G16H 50/30

(52) U.S. Cl. CPC G16H 20/00; G16H 10/60; G16H 15/00; G16H 50/20; G16H 50/30

(57) ABSTRACT

Provided herein are methods and systems for generating a recommendation, comprising receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation.

### CROSS-REFERENCE

This application claims the benefit of U.S. Provisional Application Ser. No. 63/682,184, filed Aug. 12, 2024, which is entirely incorporated herein by reference.

### SUMMARY

Provided herein, in one aspect, are methods for generating a recommendation, comprising: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation.

In some embodiments, the recommendation is directed to an improvement of the wellness state of the second wellness type.

In some embodiments, the method further comprises determining that an action based on the recommendation has been taken.

In some embodiments, the method further comprises determining an improvement of the wellness state of the second wellness type based on the taken action.

In some embodiments, the method further comprises determining an improvement of a wellness state of the first wellness type based on the improvement of the wellness state of the second wellness type.

In some embodiments, the method further comprises determining a correlation between or among the taken action, the improvement of the wellness state of the second wellness type, and the improvement of the wellness state of the first wellness type.

In some embodiments, the method further comprises concluding, based on the correlation, that the wellness state of the first wellness type is caused by the wellness state of the second wellness type, or that the wellness state of the second wellness type is not caused by the wellness state of the first wellness type.

In some embodiments, the method further comprises suggesting a diagnosis with respect to the wellness state of the second wellness type based on the concluding.

In some embodiments, the method further comprises suggesting a change to a treatment with respect to the wellness state of the first wellness type or the wellness state of the second wellness type based on the diagnosing.

Provided herein, in one aspect, are computer systems for generating a recommendation, comprising: a non-transitory memory; a processor in communication with the non-transitory memory, the processor configured to execute the following operations in order to effectuate any of the methods as described herein comprising the operations of: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation.

In some embodiments, the recommendation is directed to an improvement of the wellness state of the second wellness type.

In some embodiments, the operations further comprise determining that an action based on the recommendation has been taken.

In some embodiments, the operations further comprise determining an improvement of the wellness state of the second wellness type based on the taken action.

In some embodiments, the operations further comprise determining an improvement of a wellness state of the first wellness type based on the improvement of the wellness state of the second wellness type.

In some embodiments, the operations further comprise determining a correlation between or among the taken action, the improvement of the wellness state of the second wellness type, and the improvement of the wellness state of the first wellness type.

In some embodiments, the operations further comprise concluding, based on the correlation, that the wellness state of the first wellness type is caused by the wellness state of the second wellness type, or that the wellness state of the second wellness type is not caused by the wellness state of the first wellness type.

In some embodiments, the operations further comprise suggesting a diagnosis with respect to the wellness state of the second wellness type based on the concluding.

In some embodiments, the operations further comprise suggesting a change to a treatment with respect to the wellness state of the first wellness type or the wellness state of the second wellness type based on the diagnosing.

Provided herein, in one aspect, is a non-transitory computer-readable memory storing one or more instructions executable by one or more processors, that when executed by the one or more processors cause the one or more processors to perform processing any of the methods as described herein comprising: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation.

In some embodiments, the recommendation is directed to an improvement of the wellness state of the second wellness type.

In some embodiments, the one or more instructions further comprise determining that an action based on the recommendation has been taken.

In some embodiments, the one or more instructions further comprise determining an improvement of the wellness state of the second wellness type based on the taken action.

In some embodiments, the one or more instructions further comprise determining an improvement of a wellness state of the first wellness type based on the improvement of the wellness state of the second wellness type.

In some embodiments, the one or more instructions further comprise determining a correlation between or among the taken action, the improvement of the wellness state of the second wellness type, and the improvement of the wellness state of the first wellness type.

In some embodiments, the one or more instructions further comprise concluding, based on the correlation, that the wellness state of the first wellness type is caused by the wellness state of the second wellness type, or that the wellness state of the second wellness type is not caused by the wellness state of the first wellness type.

In some embodiments, the one or more instructions further comprise suggesting a diagnosis with respect to the wellness state of the second wellness type based on the concluding.

In some embodiments, the one or more instructions further comprise suggesting a change to a treatment with respect to the wellness state of the first wellness type or the wellness state of the second wellness type based on the diagnosing.

Provided herein, in one aspect, are methods for determining a wellness score for a wellness type of a subject comprising: obtaining clinical data associated with the subject; processing the clinical data to extract a plurality of clinical features; determining, by an algorithm, the wellness score based at least in part on the plurality of clinical features and a plurality of weights, wherein each of the weights in the plurality of weights is indicative of a level of a contribution of a corresponding clinical feature to the wellness score.

In some embodiments, the clinical data comprises one or more of biometrics measurements, clinical notes, subject input, or medical codes.

In some embodiments, the clinical data comprises text, and wherein extracting the plurality of clinical features comprises processing the clinical data with a large language model (LLM). In some embodiments, the LLM is pre-trained, trained, or fine-tuned using clinical notes.

In some embodiments, the method further comprises determining one or more additional wellness scores for one or more additional wellness types of the subject.

In some embodiments, the method further comprises aggregating the wellness score and the one or more additional wellness scores to generate an aggregate wellness score.

In some embodiments, the algorithm is a machine learning algorithm.

In some embodiments, the method further comprises training the machine learning algorithm by updating the plurality of weights.

In some embodiments, the method further comprises outputting the wellness score.

In some embodiments, method further comprises outputting a subset of clinical features having a high level of contribution to the wellness score.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a computing device; in this case, a device with one or more processors, memory, storage, and a network interface.

FIG. 2 shows a non-limiting example of a web/mobile application provision system; in this case, a system providing browser-based and/or native mobile user interfaces.

FIG. 3 shows a flow chart illustrating an example workflow according to some embodiments herein.

FIG. 4 shows a flow chart illustrating an example workflow according to some embodiments herein.

FIG. 5 shows a flow chart illustrating an example workflow according to some embodiments herein.

### DETAILED DESCRIPTION

Health-related issues common among individuals globally include diabetes, cancer, obesity, chronic disease, depression, anxiety, and substance abuse. However, health treatment is generally siloed. For example, healthcare professionals typically treat a patient's physical ailments solely based on physical data about the patient, such as blood pressure and cholesterol measurements. Patients suffering from mental illness are generally treated solely based on the patient's mental health data, elicited through psychological questionnaires, tests, or tools such as the DSM-5 (Diagnostic and Statistical Manual of Mental Disorder-5). Those who suffer from social issues, such as food insecurity and employment insecurity, are typically seen as individuals who would primarily and best be helped by social workers.

Current treatment methods are therefore focused on improvements within the same wellness type. For example, improving physical wellness by improving the person's physical wellness state; improving mental wellness by improving the person's mental wellness state; and improving social wellness by improving the person's social wellness state. However, there exists an important interplay between or among a subject's physical, mental, and social health and wellness, such that a wellness issue in one wellness type may be better addressed by treatments directed to one or more of the other wellness types.

For example, a person's mental wellness state can influence a person's physical wellness state. Prior mental trauma, especially during a subject's early ages, can lead to adverse physical health outcomes, including an increased risk of physical-related conditions such as diabetes, heart disease, and other conditions that manifest in physical and bodily symptoms. As another example, being in a constant state of mental stress or fear can trigger physical symptoms such as the upregulation of a subject's sympathetic nervous system and an increase in a subject's stress hormones like cortisol.

Moreover, a person's social wellness state can influence a person's mental wellness state or physical wellness state. For example, a subject that lives with an abusive person can lead to anxiety or depression in the subject. Further, food insecurity can cause anxiety, depression, or other mental illness in the subject. A subject's physical neighborhood and built environment, such as living in a neighborhood with poor air quality (e.g., near freeways, smog, or forest fires), may lead to physical conditions such as asthma-related illnesses or other respiratory illnesses.

As another example, a person's physical wellness state can influence a person's mental wellness state. For example, being post-partum may lead to post-partum depression. Being obese or having a large and sudden weight gain or change in physical appearance can cause isolation, depression, or other similar mental wellness issues. Being physically incapacitated or the loss of a caregiver can lead to feelings of isolation and depression.

Digital health applications have allowed a subject to receive data of a certain type (e.g., physical, mental, or social) and track improvements or changes within that same type. For example, an application that measures a subject's sleep cycle (e.g., physical data) can provide the subject with the average number of hours that the subject sleeps per night and further provide an appropriate recommendation for the subject's sleep habits. However, simply addressing an issue seen in one wellness type (e.g., a physical sleep deficiency) with a recommendation in that same wellness type (e.g., a recommendation to physically go to bed earlier) might fail to address the root cause of the issue. For example, the real cause of the physical sleep deficiency could be a mental issue (e.g., stress and anxiety) or a social one (e.g., noisy neighbors or poor sound insulation). Thus, there exists a need for an application that assesses and takes into account the interplay between or among the various wellness types and generates recommendations based on the interaction of one wellness type with another (e.g., mental with physical, social with mental, or physical with mental, etc.), and a need for an application to provide a well-rounded analysis of the subject that includes a holistic view of multiple wellness types (e.g., physical, mental, and social).

The inventive concepts described herein aim to identify the impact of a change in one or more of a person's physical, mental, or social wellness types on another wellness type to generate recommendations. The inventive concepts further include a function to collect and receive holistic information about a person, including one or more of the person's physical, mental, and social data. The data may be analyzed to determine possible correlations and causes between or among the various types of data, and a recommendation comprising information to ameliorate a physical, mental, or health issue based on the analysis may be generated and provided to a healthcare professional. For example, if a subject presents with physical symptoms such as elevated blood glucose levels, resting heart rate, blood pressure levels, respiratory rate, and the like, and the subject also has a history of underlying mental trauma (e.g., PTSD), the recommended treatment might be therapy to address deep-seated mental trauma and to avoid or manage the triggers for a PTSD episode that elicit the physical responses that the subject is presenting with. As a result of such treatment to the subject's mental wellness state, the subject's physical medical condition may improve, such as from diabetic to pre-diabetic or from high blood pressure to normal blood pressure. As another example, if a subject's physical data shows that the subject has become obese in a short period of time, and it is also known that the subject is struggling with depression or anxiety, then a treatment to the subject's depression and anxiety may ultimately improve the subject's obesity.

Thus, health and wellness is improved by integrating and analyzing the combination of one or more wellness states (e.g., physical, mental, and social), generating personalized recommendations, and facilitating information sharing with others (e.g., healthcare professionals) to mark a significant leap toward individualized and collaborative healthcare.

Described herein, in certain embodiments, are methods for generating a recommendation, comprising: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation. In some embodiments, the recommendation is directed to an improvement of the wellness state of the second wellness type. In some embodiments, the method further comprises: determining that an action based on the recommendation has been taken; determining an improvement of the wellness state of the second wellness type based on the taken action; determining an improvement of a wellness state of the first wellness type based on the improvement of the wellness state of the second wellness type; determining a correlation between or among the taken action, the improvement of the wellness state of the second wellness type, and the improvement of the wellness state of the first wellness type; concluding, based on the correlation, that the wellness state of the first wellness type is caused by the wellness state of the second wellness type, or that the wellness state of the second wellness type is not caused by the wellness state of the first wellness type; suggesting a diagnosis with respect to the wellness state of the second wellness type based on the concluding; and suggesting a change to a treatment with respect to the wellness state of the first wellness type or the wellness state of the second wellness type based on the diagnosing.

Also described herein, in certain embodiments, are computer systems for generating a recommendation, comprising: a non-transitory memory; a processor in communication with the non-transitory memory, the processor configured to execute the following operations in order to effectuate any of the methods as described herein comprising the operations of: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation. In some embodiments, the recommendation is directed to an improvement of the wellness state of the second wellness type. In some embodiments, the operations further comprise: determining that an action based on the recommendation has been taken; determining an improvement of the wellness state of the second wellness type based on the taken action; determining an improvement of a wellness state of the first wellness type based on the improvement of the wellness state of the second wellness type; determining a correlation between or among the taken action, the improvement of the wellness state of the second wellness type, and the improvement of the wellness state of the first wellness type; concluding, based on the correlation, that the wellness state of the first wellness type is caused by the wellness state of the second wellness type, or that the wellness state of the second wellness type is not caused by the wellness state of the first wellness type; suggesting a diagnosis with respect to the wellness state of the second wellness type based on the concluding; and suggesting a change to a treatment with respect to the wellness state of the first wellness type or the wellness state of the second wellness type based on the diagnosing.

Also described herein, in certain embodiments, is a non-transitory computer-readable memory storing one or more instructions executable by one or more processors, that when executed by the one or more processors cause the one or more processors to perform processing of any of the methods as described herein comprising: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation. In some embodiments, the recommendation is directed to an improvement of the wellness state of the second wellness type. In some embodiments, the one or more instructions further comprise: determining that an action based on the recommendation has been taken; determining an improvement of the wellness state of the second wellness type based on the taken action; determining an improvement of a wellness state of the first wellness type based on the improvement of the wellness state of the second wellness type; determining a correlation between or among the taken action, the improvement of the wellness state of the second wellness type, and the improvement of the wellness state of the first wellness type; concluding, based on the correlation, that the wellness state of the first wellness type is caused by the wellness state of the second wellness type, or that the wellness state of the second wellness type is not caused by the wellness state of the first wellness type; suggesting a diagnosis with respect to the wellness state of the second wellness type based on the concluding; and suggesting a change to a treatment with respect to the wellness state of the first wellness type or the wellness state of the second wellness type based on the diagnosing.

### Methods

Provided herein, in some aspects, are methods for generating a recommendation. In some embodiments, the methods for generating a recommendation may comprise: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation.

### Generating a Recommendation

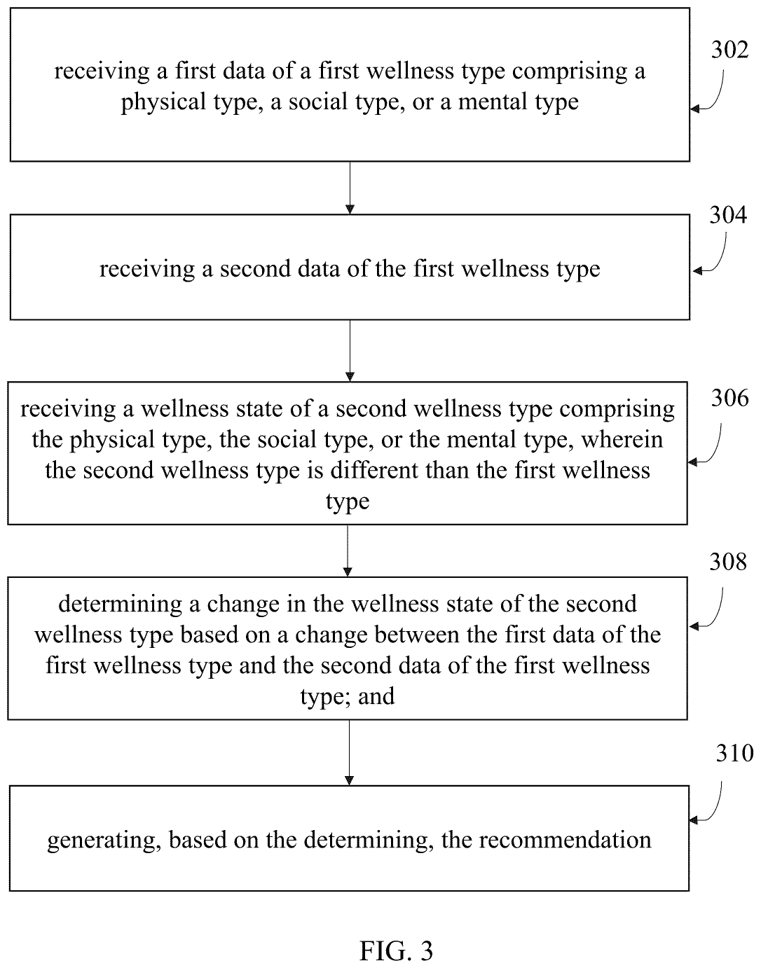

FIG. 3 illustrates an example workflow describing method operations as described herein. FIG. 3 illustrates operations 302, 304, 306, 308, and 310. Operations 302, 304, 306, 308, and 310 are non-limiting.

At operation 302, the method may comprise receiving a first data of a first wellness type. In some embodiments, the first wellness type may comprise a physical type, a social type, or a mental type. Non-limiting examples of physical, social, and mental wellness types are illustrated in the “Wellness Types” section herein. In some embodiments, the first wellness type is a physical type. Thus, in such embodiments, the first data comprises information describing or relating to physical wellness, which may be referred to herein as physical data. Non-limiting examples of physical data include resting heart rate, blood pressure, body temperature, breathing rate, blood oxygen content, blood glucose level, weight, body mass index (BMI), grip strength, flexibility, and height (see also “Data” section). In some embodiments, the first wellness type is a social type. Thus, in such embodiments, the first data comprises information describing or relating to social wellness, which may be referred to herein as social data. Non-limiting examples of social data include address, sunlight exposure, amount of social interaction, proximity to green spaces, employment status, relationship status, access to drinkable water, access to healthy food, air quality, quality of neighborhood and built environment, (see also “Data” section). In some embodiments, the first wellness type is a mental type, and the first data is of a mental type, which may be referred to herein as mental data. Non-limiting examples of mental data include brain waves, fatigue levels, motivation levels, impulsivity, moodiness, and brain chemistry (see also “Data” section).

To illustrate, at operation 302, a first physical data of a resting heart rate of 60 beats per minute (bpm) may be received.

At operation 304, the method may comprise receiving a second data of the first wellness type. In some embodiments, the first wellness type may be a physical type. Thus, in such embodiments, the first and second data would both comprise physical wellness data. In some embodiments, the first wellness type may be a social wellness type. Thus, in such embodiments, the first and second data would comprise social data. In some embodiments, the first wellness type may be a mental wellness type. Thus, in such embodiments, the first and second data would comprise mental data.

For further illustration, at operation 304, a second physical data of a resting heart rate of 120 bpm may be received.

At operation 306, the method may comprise receiving a wellness state of a second wellness type. A wellness state may comprise one or more conditions, circumstances, situations, states, positions, diagnoses, or other descriptions of a person's physical, social, or mental wellness. The second wellness type may comprise a physical type, a social type, or a mental type. The second wellness type may be different than the first wellness type of operations 302 and 304. In some embodiments, the first wellness type is a physical wellness type, and the second wellness type is a social wellness type. In some embodiments, the first wellness type is a physical wellness type, and the second wellness type is a mental wellness type. In some embodiments, the first wellness type is a social wellness type, and the second wellness type is a physical wellness type. In some embodiments, the first wellness type is social wellness type, and the second wellness type is a mental wellness type. In some embodiments, the first wellness type is a mental wellness type, and the second wellness type is a physical wellness type. In some embodiments, the first wellness type is a mental wellness type, and the second wellness type is a social wellness type. Some examples of a physical wellness state include being obese, diabetic, and hypertensive. Some examples of a social wellness state include being homeless, lacking food security, and socially isolated. Some examples of a mental wellness state include being happy, peaceful, calm, agitated, confident, satisfied, accepted, agitated, depressed, excited, fearful, angry, guilty, and ashamed.

In some examples, the subject's wellness state is directly elicited by displaying prompts to the subject. Such prompts may comprise questions regarding the subject's physical, social, or mental wellness. The subject may provide a response to such questions by selecting a predetermined textual or non-textual (e.g., icons, graphics, pictures, and sounds) response, or providing a free-form textual or non-textual response. For example, a subject may be asked how he or she is mentally feeling, and the subject may respond that she is feeling calm. In some examples, the subject's wellness state is received by parsing, capturing, or otherwise obtaining information about the subject's wellness from documentation such as doctor's reports, the subject's personal notes, written or oral accounts from third parties, or any other relevant source. For example, the subject may have been previously diagnosed with an anxiety disorder, and a copy of the subject's medical records may be scanned, photographed, or otherwise provided by or for the subject to the computer system performing the method.

In some examples, based on the subject's responses to the question prompts or the receipt of such parsed, captured, or otherwise obtained information, a baseline physical, social, and mental wellness state corresponding to an absence of the relevant physical, social, or mental wellness issue elicited from the subject or otherwise obtained about the subject from such source. For example, a subject suffering from PTSD may be determined to have a baseline mental state of “calm,” and a triggered state of “agitated and nervous.” In some examples, from time to time, the subject may be asked context-specific follow-up questions to determine the subject's current wellness state. For instance, such prompts for such follow-up questions may be displayed at certain predetermined times of the day (e.g., morning, afternoon, and night), with a fixed frequency (e.g., every hour), at or near specific locations (e.g., near the site of a tragedy) or as would otherwise make sense based on information received about the subject's physical, social, or mental wellness issues.

With reference to the illustration above in which a first and second resting heart rate data is received, at operation 306, a mental wellness state of being calm may be received.

At operation 308, the method may comprise determining a change in the wellness state of the second wellness type based on a change between the first data of the first wellness type and the second data of the first wellness type. With reference to the scenario illustrated above in which the first data and the second data are resting heart rate data and the wellness state that is received is a calm mental state, at operation 308, a change from a calm mental state to an agitated mental state may be determined based on a change between the first resting heart rate of 60 bpm and the second resting heart rate of 120 bpm. Thus, in some examples, the change in one symptomatic wellness type (e.g., physical) for which data is received (e.g., heart rate data), is used to inform, infer, predict, or otherwise determine a change in another wellness type (e.g., mental). In some examples, data about such other wellness type is also received, and such determination of change in such other wellness type is also based on such data about such other wellness type. In some examples, such data about such other wellness type is qualitatively or quantitatively less than the data about the first wellness type. In some examples, such data about such other wellness type is chronologically more distant than the data about the first wellness type, that is, such data was measured or recorded less recently than the data about the first wellness type. In some examples, such data about such other wellness type is technically more difficult or inconvenient to measure or obtain than the data about the first wellness type.

In some examples, the determination of such change in the second wellness type comprises asking or presenting the subject with questions to identify, confirm, or rule out the cause or causes of such change. In some examples, the questions may be asked via a software application or other computer program, such as a mobile application or a web-based application. Such questions may be general or context-specific, and the responses to such questions may be used to confirm the presence or absence of common causes for such change, such as, in the case of a sudden increase in resting heart rate, whether the subject is: feeling chest pains, experiencing shortness of breath, having cold sweats, exercising or engaging in other physical activity, or is experiencing other physical symptoms (which responses thereto could be used to confirm the presence or absence of a physical wellness issue such as a heart attack); replaying a traumatic episode in their minds or experiencing other mental symptoms (which responses thereto could be used to confirm the presence or absence of a mental wellness issue such as PTSD); in an altercation, experiencing an episode of domestic or other social abuse, being socially alienated or victimized (e.g., racism), or experiencing or witnessing a crime or other violent event (which responses thereto could be used to confirm the presence or absence of a social wellness issue such as exposure to an unhealthy social and community context or being domiciled in a poor neighborhood or built environment). The subject may also be asked questions based on the subject's physical, mental, or social information. For example, if the subject provided information regarding a history of physical, mental, or social disorders, or if the subject is determined (for example, through examination, relation, prediction or other means based on the subject's physical, mental, or social information) to be susceptible to certain disorders that manifest or otherwise present symptoms consistent with the change between the first data and the second data, the subject may be asked to confirm the presence of absence of a current episode or manifestation of such disorder.

At operation 310, the method may comprise generating a recommendation. The generating the recommendation may be based on the determining of operation 308. In some examples, the recommendation is directed to improving the subject's mental state. In other examples, the recommendation is directed to improving the subject's physical state. In further examples, the recommendation is directed to improving the subject's social state. Non-limiting examples of recommendations are provided in the “Recommendations” section herein.

With further reference to the scenario described above in which a change in a subject's mental state (e.g., from calm to agitated) is determined based on a change in resting heart rate from 60 bpm to 120 bpm, a recommendation to improve the subject's agitated mental state may include mental health suggestions such as therapy, medication, or both.

In some examples, the recommendation is generated using machine learning techniques described herein. For example, the recommendation may be generated using supervised, unsupervised, semi-supervised, reinforcement learning, transduction, “learning to learn,” or any other approach. Such approaches may involve identifying and recognizing patterns in existing data to facilitate making recommendations to improve the wellness state of the second wellness type when provided with the first data and the second data of the first wellness type (and in some examples, data of the second wellness type). Machine learning may also provide deductive or abductive inference based on real or simulated data. As further described below, machine learning may include training a machine learning model and validating one or more aspects of the machine learning model.

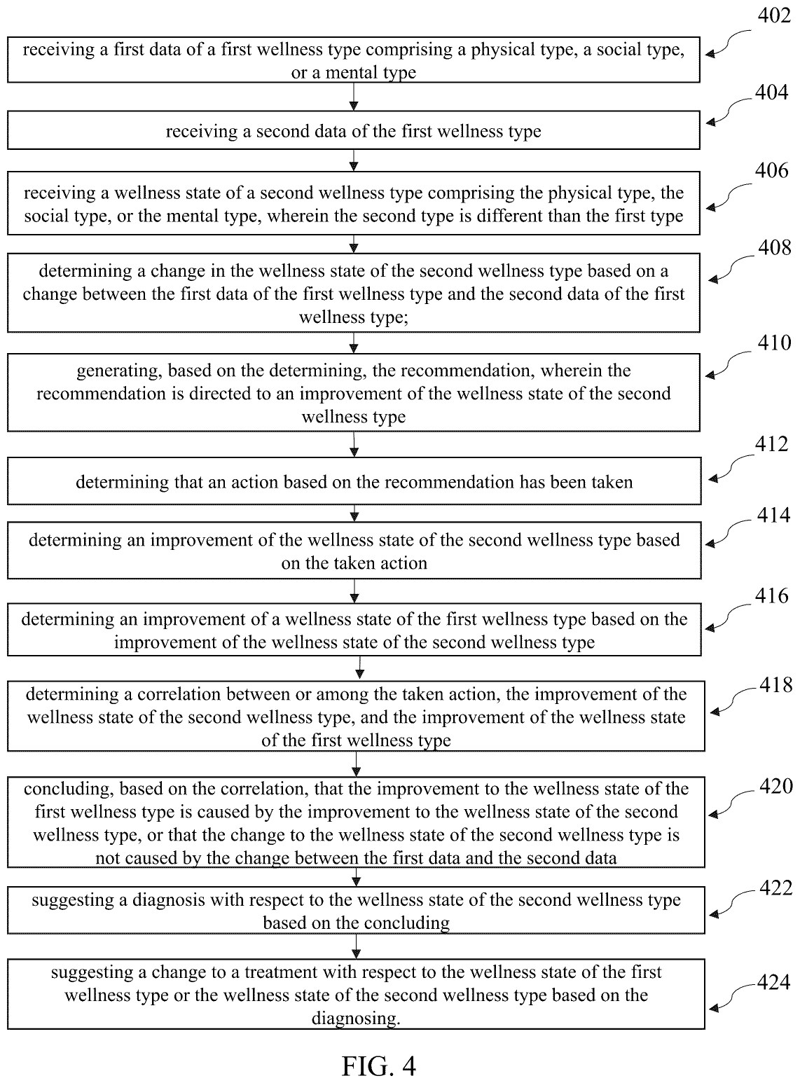

FIG. 4 illustrates the example workflow depicted in FIG. 3 with additional method operations as described herein. As such, in some embodiments, operations 402, 404, 406, and 408 of FIG. 4 may comprise operations 302, 304, 306, and 308, respectively, of FIG. 3, incorporated herein by reference. FIG. 4 further illustrates operations 410, 412, 414, 416, 418, 420, 422, and 424. Operations 402, 404, 406, 408, 410, 412, 414, 416, 418, 420, 422, and 424 are non-limiting.

Operation 410 of FIG. 4 may comprise operation 310 of FIG. 3. In Operation 410, the recommendation may be directed to an improvement of the wellness state of the second wellness type. In one example, the first wellness type is a physical wellness type, and the second wellness type is a mental wellness type. A recommendation to improve the mental wellness state, such as being agitated, may, for example, include a suggestion to begin breathing exercises or meditation, take a walk, play with a pet, use mental health resources, commence a psychotherapy session, seek medical help, or a reminder to take one's prescribed medication. In some examples, the recommendation may be directed to improving a wellness state of a wellness type (e.g., mental) that is different from the wellness type corresponding to the received data (e.g., physical). Thus, in the example above in which a subject's mental state is determined to change from calm to agitated on the basis of the increase in heart rate, the recommendation would be directed to improving the subject's agitated mental state, notwithstanding or in spite of the fact that the subject presented with physical data (e.g., an elevated heart rate from 60 bpm to 120 bpm) that may also be consistent with physical symptoms (e.g., a mild or impending heart attack). Thus, instead of a recommendation to improve the subject's physical symptoms on the basis of the increased heart rate, such as to put oneself in a physical recovery position, the recommendation may instead be to start a breathing exercise or meditation to calm the mind.

At operation 412, the method may comprise determining that an action based on the recommendation of operation 410 has been taken. Such determination may be directly made based on input provided by the subject regarding the action, or indirectly by analyzing information about the subject provided by sources other than the subject (e.g., cameras, monitors, and sensors) and inferring that the action has been taken. In one example, the subject may be asked whether the subject performed the recommended action, and the subject may be asked to affirmatively respond with a yes or a no. If the subject responds with a “yes,” then it is determined that the subject performed the recommended action. In an example where the recommended action may be to perform breathing exercises, the subject may be asked “did you perform the breathing exercises?” In another example, the subject may be asked to input information on the action that the subject took or did not take. For example, after the recommendation is generated and provided to the subject, the subject may be asked to “enter the action taken here.” In a further example, the subject may be monitored by a sensor, such as a sensor included in a hardware device such as a camera, watch, smart phone, a ring, or an implanted device such as a brain chip. The sensor may capture data on the subject, which may be transmitted or otherwise provided to a computer system performing the method described herein. For example, sensors may capture and transmit the subject's breathing rate, which can be used to determine whether or not the subject performed the recommended action of breathing exercises. Thus, if the recommendation was to perform slow deep breaths for one minute, and the subject's breathing rate slowed down by an expected or statistically significant amount (e.g., a predetermined number of standard deviations away from the subject's recent average resting breathing rate) during such one minute period, then it may be determined that the subject has performed the recommended action.

At operation 414, the method may comprise determining an improvement of the wellness state of the second wellness type of operation 410 based on the taken action of operation 412. In an example where the wellness state of the second wellness type (e.g., mental) is the state of being mentally agitated, and where the recommendation is to perform breathing exercises, a determination may be made as to whether or not such mental agitation has been improved by the performance of the recommended breathing exercises. In one aspect, the subject may provide feedback or a rating indicating how well the recommendation worked for the subject in improving the subject's wellness (or wellness state at issue) of the second wellness type, or how much the recommendation has improved or worsened the subject's wellness (or wellness state at issue) of the second wellness type. For example, a scoring system may be made available to the subject where a score of +100 indicates that the recommendation worked superlatively well or most improved the wellness (or wellness state at issue) of the second wellness type, a score of 0 indicates that the recommendation did not change the subject's wellness (or wellness state at issue) of the second wellness type, and a score of −100 indicates that the recommendation worked superlatively poorly or most worsened the wellness (or wellness state at issue) of the second wellness type. An overall score of a particular wellness state of the second wellness type (e.g., mental agitation) may be kept over time by summing the individual scores of the same wellness states at issue of the second wellness type (e.g., mental agitation score 1+mental agitation score 2+mental agitation score 3+ . . . ), and such score may be indicative of the subject's overall wellness e.g. with respect to such wellness states at issue of the second wellness type (e.g., overall mental agitation score over a time period). In some examples, an overall score for wellness of the second wellness type may be obtained by summing the individual scores of different wellness states of the second wellness type (e.g., depression score 1+mental agitation score 1+depression score 2+mental agitation score 2+depression score 3+mental agitation score 3+ . . . ). In some examples, the foregoing scores may be normalized to a numerical scale, such as 0 to 1000 point scale, with 0 indicating the poorest health and 1000 indicating the best health. In some examples, the scores of the three wellness types are summed to indicate the subject's overall physical, social, and mental wellness. Such overall wellness score may be normalized to a scale, scale, such as 0 to 1000 point scale, with 0 indicating the poorest health and 1000 indicating the best health. Thus, when the subject indicates an improvement to a wellness type(s) or a wellness state(s), the subject's wellness score in the corresponding wellness type(s) or wellness state(s) would increase, bringing the score closer to the value indicating best health, for example 1000. Similarly, when the subject indicates a deterioration in a wellness type(s) or a wellness state(s), the subject's wellness score in the corresponding wellness type(s) or wellness state(s) would decrease, bringing the score closer to the value indicating poorest health, for example, 0. The points may be displayed to the user as a barometer of the user's health in one or more wellness type(s) or state(s). The points may be displayed to the user on a graphical user interface (GUI). The subject's score may be updated each time the subject indicates an improvement or worsening in wellness, or updated only after statistically significant reporting by the subject. Such a scoring system may gamify wellness and motivate the subject to improve on aspects of the subject's wellness in an overall manner or with respect to particular wellness states.

In some examples, the subject may be asked to describe the effect of the recommendation on the wellness state of the second wellness type. In some examples, the subject may be asked whether the subject feels better after performing the recommended action and to affirmatively respond with a yes or a no. If the subject responds with a “yes,” then it is determined that the wellness state of the second wellness type improved based on the performance of the recommended action. In an example where the recommended action may be to perform breathing exercises, the subject may be asked “do you feel better after performing the breathing exercises?” In another example, the subject may be asked to input information as to the effect of the action that the subject took or did not take. For example, after the action is determined to have been taken by the subject, the subject may be asked to “describe how you feel after performing the action” or to describe the effect of the action on the second wellness state (e.g., mental agitation). If the subject responds that the subject feels better after performing the recommended action or provides a positive description of the effect of the recommended action on the wellness state at issue (as may be determined using artificial intelligence techniques, such as a large language model), then it is determined that the wellness state of the second wellness type is improved based on the taken action. In a further example, the subject may be monitored by a sensor, such as a sensor included in a hardware device such as a camera, watch, smart phone, ring, or an implanted device such as a brain chip. The sensor may capture data on the subject, which may be transmitted or otherwise provided to the computer system performing the method described herein. For example, sensors may capture the presence of certain brain waves, a smile, a reduction in tension in the facial muscles, or other data that correlate in a statistically significant manner to an improvement of an agitated mental state.

At operation 416, the method may comprise determining an improvement of a wellness state of the first wellness type of operation 402 and 404 based on the improvement of the wellness state of the second wellness type of operation 414. In the example where the first wellness type is a physical type, the second wellness type is a mental type, and the improvement of the wellness state of the second wellness type is an improvement in the subject's mental state from an agitated mental state to a calm mental state, such improvement in the subject's mental state e.g. may be used to determine an improvement in the subject's physical wellness state, and more specifically, the wellness state of the relevant physical aspect (e.g., the subject's heart) aspect subject's heart. In some examples, the improvement in the subject's wellness state of the second wellness type (e.g., from agitated to calm) may prompt or trigger a collection or analysis of information relating to the relevant wellness state of the first wellness type (e.g., cardiac wellness). Information that relates to or is derived from the same source for the first data of the first wellness type or the second data of the first wellness type may be deemed to be relevant. For example, if the first and second data of the first wellness type is heart rate data, a relevant wellness state of the first wellness type may be the wellness state of the heart, as heart rate relates to or is derived from the heart. Referring to the earlier example, after the subject performs the recommended action (e.g., breathing exercises) resulting in an improvement to the subject's agitated mental state is determined (e.g., by a response from the subject indicating that the subject now feels calm), the subject's heart rate may be taken and compared to the subject's earlier heart rate prior to the breathing exercises. For example, after the breathing exercises and the calming of the subject's mental state, the subject's heart rate may fall in a statistically significant manner (e.g., from 120 bpm to 60 bpm), which may indicate a general improvement to the subject's physical wellness state, and more particularly, an improvement to the wellness state of the subject's heart. Generally speaking then, after it is determined that the recommended action results in an improvement to the wellness state of the second wellness type, in some examples, e.g., relevant information of the first wellness type (e.g., heart rate after breathing exercises) may be collected and compared to similar information of the first wellness type obtained prior to the performance of the recommended action (e.g., heart rate at the time of mental agitation) to determine if there has been an improvement to the wellness state of the first wellness type.

At operation 418, the method may comprise determining a correlation. The correlation may be between or among one or more of: (i) the taken action of operation 412; (ii) the improvement of the wellness state of the second wellness type of operation 414; and (iii) the improvement of the wellness state of the first wellness type of operation 416. To illustrate with an example, a correlation may be determined between or among one or more of: (a) the subject practicing breathing exercises; (b) the improvement of the subject's agitated mental state; and (c) the improvement of the subject's cardiac wellness state. Such correlation may be measured using statistical techniques, such as the Pearson correlation, Kendall rank correlation, the Spearman Correlation, the Point-Biserial correlation, the Correlation Ratio, Cramer's V, and others. The strength of a correlation may be measured using a correlation coefficient that varies between +1 and −1. A value of ±1 may indicate a perfect degree of association between the two variables. As the correlation coefficient value goes towards 0, the relationship between the two variables will be weaker. The direction of the relationship may be indicated by the sign of the coefficient; a +sign indicates a positive relationship and a −sign indicates a negative relationship. In some examples, the correlation that is determined between or among the foregoing variables (i)-(iii) may be a positive correlation, in which the taken action positively relates to an improvement of the wellness state of the second wellness type, and the improvement of the wellness state of the second wellness type positively relates to an improvement of the wellness state of the first wellness type. In some examples, such correlations are strongly positive and have a value close to the value that indicates a perfect degree of positive association between or among the variables, for example, correlation coefficient value, r, of 0.7 and above.

At operation 420, the method may comprise concluding a causal relationship based on the correlation of operation 418. In some examples, the conclusion (based on the correlation) is that the improvement to the wellness state of the first wellness type is caused by the improvement to the wellness state of the second wellness type. In some examples, the conclusion (based on the correlation) is that the change to the wellness state of the second wellness type is not caused by the change between the first data of the first wellness type and the second data of the first wellness type. To illustrate using the mental agitation and cardiac wellness example discussed above, the method may conclude that the improvement to the subject's physical wellness state (e.g., lower resting heart rate indicative of improved cardiac wellness) is caused by the improvement to the subject's mental wellness state (e.g., from agitated to calm). The method may also conclude that the change to the subject's mental wellness state (e.g., from calm to agitated) is not caused by the change in the heart rate (e.g. from 60 bpm to 120 bpm).

In some examples, such conclusion may be reached when there is a strong positive relationship (e.g., r value >0.7) between the improvement to the wellness state of the second wellness type and the improvement to the wellness state of the first wellness type, and a weak relationship (e.g., 0<r<0.2) between the improvement to the wellness state of the first wellness type and the improvement to the wellness state of the second wellness type.

At operation 422, the method may comprise suggesting a diagnosis. The diagnosis may be based on the concluding of operation 420. The diagnosis may be with respect to the wellness state of the second wellness type. For example, based on the conclusion that the improvement to the wellness state of the first wellness type (e.g. lower resting heart rate indicative of improved cardiac wellness) is caused by the improvement to the wellness state of the second wellness type (e.g., agitated to calm mental state), a suggested diagnosis may be that the subject suffers from an anxiety disorder, such as general anxiety disorder or a panic disorder, mental stress, or PTSD. As such, even though the subject presented with a symptom (e.g., elevated heart rate) of a first wellness type (e.g., physical), the diagnosis is not made in the first wellness type (e.g., physical) but in the second wellness type (e.g., mental).

At operation 424, the method may comprise suggesting a change to a treatment. The change to the treatment may comprise stopping, beginning, modifying, or switching a treatment. The suggesting a change to a treatment may be based on the diagnosing of operation 422. The suggesting a change to a treatment may be with respect to the wellness state of the first wellness type. The suggesting a change to a treatment may be with respect to the wellness state of the second wellness type. For example, the subject may have been previously prescribed a medication, such as beta blockers, to reduce the subject's high heart rate. But based on the suggested diagnosis, a change to such treatment may be suggested, such that the suggested treatment is instead or additionally directed to improving the subject's wellness state (e.g., anxiety, mental stress, and PTSD) of the second wellness type (e.g., mental), rather than the subject's wellness state (e.g., tachycardia) of the first wellness type (e.g., physical).

### Subjects

The methods disclosed herein may relate to a human subject. The subject may be male. The subject may be female. The subject may be an adult (e.g., 18 years of age or older). The subject may be a child (e.g., less than 18 years of age).

In some embodiments, the subject may be a human whose data is being provided to the application. The subject may be the user of the application. In some examples, a third person (such as a healthcare professional, an operator, or a caretaker) may provide data on behalf of the subject whose wellness is at issue (in which case the latter would be the subject).

### Wellness Types

The methods disclosed herein may relate to a wellness type. The wellness types may comprise a physical wellness type, a social wellness type, or a mental wellness type. In some embodiments, the wellness type is a physical wellness type. Physical wellness encompasses the overall health and well-being of an individual's physical body and its systems. This may include various facets such as nutrition, exercise, rest, and overall bodily function. Physical wellness may not be solely about the absence of physical illness or disease, but rather the presence of physical vitality and optimal physical functioning. Physical wellness may relate to the health of the physical body of a subject, such as a subject's bodily movement, physical/chemical structure, and measurements. A subject's physical wellness may be further described by one or more physical wellness states, such as the subject being obese, diabetic, tachycardic, etc. Physical wellness may be evaluated through a multitude of factors. For example, nutrition can be assessed by considering the quality and balance of a subject's diet, including intake of essential nutrients, vitamins, and minerals. Also, exercise may be evaluated by considering the frequency, intensity, and type of physical activity undertaken by the subject, as well as adherence to recommended exercise guidelines. Other factors, like rest and sleep patterns may be examined, including the duration and quality of sleep, as well as the subject's ability to rest and recuperate adequately. In regard to physical wellness, the subject's medical health is monitored, including the presence of any chronic or acute physical illnesses, diseases, or conditions. The subject's engagement in preventive health measures, such as vaccinations, screenings, and regular health check-ups, may also be considered. Furthermore, the subject's use of substances such as tobacco, alcohol, or drugs and its impact on their physical well-being may be assessed. Environmental factors play a crucial role, including the influence of the subject's physical environment on their health, such as exposure to pollutants, toxins, and hazards. Body composition may be evaluated, including the subject's body mass index (BMI), body fat percentage, muscle mass, and overall body composition. Functional ability may also be assessed, including the subject's ability to perform activities of daily living, mobility, and functional independence. A subject's physical wellness may also be broadly characterized by various states or conditions, such as a state or condition of optimal health, chronic illness, disability, or injury.

In some embodiments, the wellness type is a social wellness type. Social wellness may relate to the health of a subject's social relationships, such as a subject's relationships with family, friends, and partners. Social wellness may also relate to the health of a subject's social well-being and feeling of belongingness in society. Social wellness may be evaluated based on social determinants of health, such as a subject's physical environment and living conditions, including a subject's neighborhood and built environment, place of living, city, state, and country; economic stability; social and community context, including a subject's relationship with co-habitants, such as family, spouse, or a roommate; employment; neighborhood socioeconomic characteristics; education access and quality; food insecurities; safety; environmental conditions; access to clean air and water; healthcare access and quality; housing; access to care; health literacy; and social influence. A subject's social wellness may be further described by one or more social wellness states, such as the subject's homeless, joblessness, or poverty. In some embodiments, the wellness type is a mental wellness type. Mental wellness encompasses the state of well-being concerning an individual's psychological and emotional health. It may involve not only the absence of mental illness but also the presence of positive psychological functioning and emotional resilience. Mental wellness may be closely tied to various aspects of an individual's inner world and external environment, and may encompass an individual's mental internalization and reconciliation of personal experiences and social interactions. Mental wellness may be delineated by the quality of an individual's emotional experiences and their ability to regulate emotions effectively. It may relate to one's capacity to cope with stress, adapt to adversity, and maintain a sense of balance and inner peace. Additionally, mental wellness may be influenced by the quality of an individual's relationships, including those with family members, friends, colleagues, and broader social networks. Positive social connections and support systems contribute significantly to mental well-being, fostering a sense of belonging and emotional security. Mental wellness may relate to a subject's brain or brain function, which may be quantified, analyzed, or influenced by the balance of certain chemicals and hormones in the brain, such as serotonin, dopamine, norepinephrine, endorphins, and oxytocin. Mental wellness may also relate to a subject's mental health and mental well-being. Mental wellness may be further described by one or more mental wellness states, such as the subject being depressed, bipolar, or schizophrenic.

### Data

The methods may relate to receiving data. In some embodiments, the data corresponds to a wellness type as described herein. For example, the data may comprise a physical wellness type, a social wellness type, or a mental wellness type.

In some embodiments, physical data may relate to a subject's weight. The physical data may be a subject's weight. The physical data may be a subject's weight in pounds (lbs.) or kilograms (kg). The physical data may relate to a subject's body composition, such as a subject's body mass index (BMI), body fat percentage, or muscle mass. The physical data may be a subject's height, for example, in inches and feet. The physical data may be a subject's width. The physical data may be a subject's waist circumference. The physical data may be a subject's density.

In some embodiments, physical data may relate to a subject's heart. The physical data may be a subject's heart rate, including resting heart rate. The physical data may be a subject's pulse rate. The heart rate or pulse rate may be measured in beats per minute (bpm). The physical data may be a subject's blood pressure. The blood pressure may be measured in milliliters of mercury. The physical data may relate to a subject's cardiac output. The cardiac output may be measured in liters per minute. The physical data may relate to a subject's stroke volume. The stroke volume may be measured in milliliters per beat.

In some embodiments, physical data may relate to a subject's lungs. The physical data may be a subject's respiration rate or breathing rate. The respiration rate or breathing rate may be measured in breaths per minute. The physical data may be a subject's lung capacity. The lung capacity may be measured in liters.

In some embodiments, physical data may relate to a subject's temperature (e.g., body temperature). The body temperature may be obtained orally, rectally, by ear, by skin, or by any other body temperature measurement method. The body temperature may be measured in degrees Fahrenheit or in degrees Celsius.

In some embodiments, physical data may relate to a subject's eyes. The physical data may be obtained using a visual acuity measurement test, eye movement test, glaucoma test, a retinal exam, depth perception test, slit lamp exam, or an intraocular pressure test. The vision of the subject may be measured in meters or feet. The eye movement of the subject may be measured in saccade duration, revisited fixation duration, number fixation, or pupil size. The eye pressure of the subject may be measured in millimeter of mercury.

In some embodiments, physical data may relate to a subject's strength. The physical data may be a subject's grip strength. The physical data may be a subject's ability to lift objects, for example, the subject's ability to deadlift a certain amount of weight. This weight may be measured in pounds or kilograms. The physical data may relate to a subject's endurance. The subject's endurance may be measured in heart rate, blood pressure, respiratory rate, and breathlessness. The physical data may relate to a subject's speed. For example, the physical data may be the time it takes a subject to run a distance, for example, in meters or in miles.

In some embodiments, physical data may relate to a subject's flexibility. For example, physical data may be a measurement of a subject's sit and reach. In some embodiments, physical data may relate to a subject's balance. In some embodiments, physical data may relate to a subject's dexterity. The subject's dexterity may be measured in hand manipulation (rotation and shift) and functional tripod grasp patterns.

In some embodiments, physical data may relate to a subject's hormone levels. Non-limiting examples of hormones that may be used as physical data include cortisol, testosterone, growth hormone, estrogen, adrenaline, prolactin, progesterone, insulin, and serotonin. Hormones, including cortisol (e.g., a stress hormone) and thyroid hormones, can impact a subject's overall health. The hormone levels of the subject may be measured in nanograms per milliliter, nanograms per deciliter, or picograms per milliliter. Abnormalities or changes in a subject's hormone levels may be indicative of a certain health condition or contribute to symptoms such as anxiety or depression.

In some embodiments, physical data may relate to biological markers or biomarkers. Biomarkers may include proteins, metabolites, lipids, or nucleic acids. Proteins may include antibodies, enzymes, hormonal proteins, structural proteins, storage proteins, transport proteins, contractile proteins, or a combination thereof. Nucleic acids may include DNA. Nucleic acids may include RNA. The physical data may relate to a change or abnormalities in a subject's biomarkers. The physical data may relate to a mutation in a subject's nucleic acids, such as a deletion, a duplication, an inversion, or a translocation. The physical data may relate to the expression (e.g., upregulation or downregulation) of a subject's genes.

In some embodiments, physical data may relate to a subject's quality of sleep. The physical data may be a number of hours that the subject sleeps each day. For example, the subject may sleep one or more hours of sleep each day. Hours of sleep may be transmitted and received through a sleep monitoring device, such as a watch or a ring. The physical data may be count of apneas and hypopneas, oxygen levels, brainwave activity, airflow, heart rate, body movements, breathing rate, and/or sleep time in light sleep versus deep sleep. The sleep quality of a subject may be measured in breaths per minute, episodes per hour, cubic feet per minute, beats per minute, hours, or electrical activity in the brain.

In some embodiments, physical data may relate to a subject's blood oxygen levels or blood oxygen saturation. A subject's blood oxygen levels may be referred to as the amount of oxygen that a subject has circulating in the subject's blood. A subject's blood oxygen levels may be measured in a percentage. Blood oxygen levels may be transmitted and received through a wearable monitoring device, such as a watch or a ring.

In some embodiments, social data may relate to a subject's living environment. A subject's living environment may include whether the subject lives with one or more persons or animals. For example, the subject may live with a spouse, a boyfriend, a girlfriend, a partner, a roommate, or a family member. The subject may live with an abusive person. The subject may live with a physically or a verbally abusive person. The subject may live with a non-abusive person. A subject's living environment may include whether the subject has access to healthy food providers or food distribution centers, for example, a subject may be a number of miles from a healthy food source. A subject's living environment may include whether the subject lives in an area with sufficient or poor air quality. The subject may live in an area with poor air quality. For example, the subject may live in proximity to a factory or a coal plant that emits air pollution. A subject's living environment may include a subject's proximity to shrubbery and vegetation, also known as “green spaces.” A subject's living environment may include whether the subject is exposed to noise pollution. For example, a subject may be exposed to noise pollution if the subject's lives near a freeway, an airport, or a landfill. As such, a subject's proximity to cars, planes, and other motor vehicles may contribute to the subject's exposure to noise pollution in the subject's living environment. The subject's living environment may include exposure to crime. For example, the subject may live in an area that is high in crime. The subject may live in an area that is low in crime. The subject's living environment may include the subject's access to clean parks and schools. The subject's living environment may include the subject's access to sunlight. For example, the subject live on the top floor of an apartment complex and have readily access to sunlight. The subject may live on the bottom floor of an apartment complex and have limited access to sunlight.

In some embodiments, social data may relate to a subject's employment status. The social data may be that the subject is unemployed. The social data may be that the subject is employed. The subject may be employed at one or more jobs.

In some embodiments, social data may relate to a subject's social tendencies. For example, the subject may have a job that requires constant social interaction. The subject may have a job that requires zero social interaction. The subject may live in an area that encourages social interaction. The subject may live close to friends and family, which may encourage social interaction. The subject may live in an area that does not encourage social interaction. The subject may live far away from family and friends, which may discourage social interaction. The subject may have an abundance of family and friends. The subject may have no family or friends. The subject may have high sociability. The subject may have feelings of loneliness.

In some embodiments, social data may relate to a subject's exposure to sun. A subject's exposure to sun may be measured by the number of hours a day a subject gets exposure to sun, for example, the subject may have one or more hours of sun exposure per day.

In some embodiments, mental data may relate to a subject's brain chemistry. The mental data may include a level of neurotransmitters in a subject's brain. The mental data may include a presence or an absence of neurotransmitters in a subject's brain. Non-limiting examples of neurotransmitters include serotonin, acetylcholine, dopamine, glutamate, GABA, epinephrine, and norepinephrine. The mental data may include a level of endorphins in a subject.

In some embodiments, mental data may relate to a subject's emotional well-being. The subject's emotional well-being may include a subject's mood, level of stress, or overall emotional well-being. The emotional well-being may include fluctuations in the subject's mood or level of stress.

In some embodiments, mental data may relate to data related to subject's cognitive abilities. The subject's cognitive abilities may include the subject's levels or spans of memory or attention. The subject's cognitive abilities may include the subject's problem-solving skills or other cognitive processes.

In some embodiments, mental data may relate to a subject's behavioral patterns. For example, the subject's behavioral patterns may include observations of the subject's behavior and records of that behavior. The subject's behavioral patterns may include the subject's habits or routines, which may provide insights into the subject's mental well-being.

In some embodiments, mental data may relate to therapeutic interventions of the subject. For example, mental data may include records of therapeutic interventions of the subject, such as counseling sessions or medication record/management, which may provide insight into a subject's mental well-being.

In some embodiments, mental data may relate to a subject's brain structure. For example, a computed tomography (CT) scan may be used to obtain structures of a subject's brain. As another example, a magnetic resonance imager (MRI) may be used to obtain images of a subject's brain. Structures of a subject's brain may help identify injuries the subject may have.

In some embodiments, mental data may relate to a subject's brain waves. For example, an electroencephalography (EEG) may be used to record electrical waves in a subject's brain. A positron emission tomography (PET) scan may be used to analyze a subject's brain when the subject performs certain tasks.

The methods disclosed herein may relate to receiving one or more data. In some embodiments, one or more data is received. The one or more data may comprise a wellness type, for example, a physical wellness type, a social wellness type, or a mental wellness type.

### Wellness States

The methods may relate to receiving a wellness state. In some embodiments, the wellness state corresponds to a wellness type as described herein. For example, the wellness state may comprise conditions, circumstances, situations, states, positions, diagnoses, or other descriptions of a person's physical, social, or mental wellness.

A physical wellness state may relate to or be described by an injury such as a broken bone, a fractured bone, a dislocated bone, a ligament tear, a ligament sprain, a tendon tear, a muscle laceration, or a muscle strain. An injury to the bone may be a break or a fracture. For example, the subject may have a broken bone, such as a broken clavicle, arm, wrist, hip, ankle, foot, toe, hand, finger, leg, rib, collarbone, femur, spine, tailbone, pelvis, or elbow. In some embodiments, the subject may have a fractured bone, such as a fractured clavicle, arm, wrist, hip, ankle, foot, toe, hand, finger, leg, rib, collarbone, femur, spine, tailbone, pelvis, or elbow. The subject may have one or more broken or fractured bones. The injury to the bone may be a dislocation. For example, the subject may have a dislocated shoulder, finger, elbow, or hip. In some embodiments, the subject may have an injury to a muscle. For example, a subject may have a laceration, contusion, a degenerative disease, or a strain to a muscle. In some embodiments, the subject may have an injury to a ligament. The injury to the ligament may include a sprain. For example, the subject may have a sprained ankle, wrist, knee, thumb, or other joint in the body. The injury to the ligament may include a tear of a ligament. In some embodiments, the subject may have an injury to a tendon. The injury to the tendon may include a tear to a tendon.

A physical wellness state may relate to or be described by a disorder or a disease. For example, the disorder or disease may include diabetes (type 1, type 2, gestational, chronic, acute, etc.), a respiratory disorder, a neurological disorder, an autoimmune disorder, or a cancer.

A physical wellness state may relate to or be described by being obese or overweight. A physical wellness state may be being underweight or anorexic.

A physical wellness state may relate to or be described by a respiratory disorder. The respiratory disorder may relate to an abnormal breathing or aspiration rate. The respiratory disorder may include asthma, chronic obstructive pulmonary disease (COPD), bronchitis (chronic or acute), emphysema, lung cancer, cystic fibrosis, pneumonia, pleural effusion, or COVID (coronavirus disease). The lung disease may comprise lung cancer. The lung cancer may be stage I, II, III, or IV lung cancer.

A physical wellness state may relate to or be described by a cancer. Non-limiting examples of cancers include pancreatic cancer, prostate cancer, myeloma, thyroid cancer, breast cancer, colorectal cancer, kidney cancer, bladder cancer, brain cancer, liver cancer, lung cancer, leukemia, and stomach cancer. The cancer may be a stage of a cancer. For example, the cancer may be stage I cancer, stage II cancer, stage III cancer, or stage IV cancer.

A physical wellness state may relate to or be described by a neurological disorder. The neurological disorder may include Parkinson's disease, Alzheimer's disease, multiple sclerosis, Huntington's disease, Amyotrophic lateral sclerosis, stroke, epilepsy, a disorder related to a brain injury, a disorder related to a spinal cord injury. Bell's palsy, or dementia.

A physical wellness state may relate to or be described by an autoimmune disease. The autoimmune disease may include lupus, thyroiditis and graves' disease, celiac disease, inflammatory bowel disease, multiple sclerosis, psoriasis, type 1 diabetes, or rheumatoid arthritis.

A social wellness state may relate to or be described by a subject's housing status; employment status (such as being employed or unemployed, or holding one or more jobs at a time); social circle (including the size and quality of the social circle); quality of life; economic stability; access to education (including the quality of such education); access to healthcare (including the quality of such healthcare); neighborhood and built environment (including the quality of such neighborhood and built environment).

A social wellness state may relate to or be described by a subject's experience with social hardship, such as social injustice, a poor living environment, or prejudice. For example, the subject may face discrimination or prejudice based on the subject's age, disability, ethnicity, gender, gender identity or expression, genetic information, national origin, race, religion, sex, or sexual orientation.

A social wellness state may relate to or be described by a subject's living conditions, such as the quality of air and water in the neighborhood, access to healthy foods, proximity to green spaces, and level of crime in the neighborhood.

A mental wellness state may relate to or be described by a presence or an absence of a mental illness or disorder, such as anxiety disorder, panic disorder, social anxiety disorder, separation anxiety disorder, depression (acute, chronic, etc.), suicidal tendencies, bipolar disorder, PTSD, schizophrenia an eating disorder (anorexia, bulimia, etc.), a developmental disorder, or a neurodevelopmental disorder (autism, attention deficit hyperactivity disorder, etc.). In some embodiments, mental wellness states may be measured and quantified. For example, depression may be measured by a PHQ-9 (Patient Health Questionnaire-9), and anxiety may be measured by the GAD-7 (General Anxiety Disorder-7 Item) scale.

However, not all wellness states are negative. Wellness states can also be positive. For example, mental wellness states may relate to or be described by excitement, delight, astonishment, happiness; and feeling pleased, content, relaxed, calm, enthusiastic, determined, inspired, active, alert, proud, attentive, focused, relieved, positive, satisfied, motivated, joyful, grateful, and accepted.

A mental wellness state may be described in accordance with the DSM-5. The DSM-5 is the 5^{th} edition of a reference book on mental health and brain-related conditions and disorders. The DSM-5 aims to provide highly detailed definitions of mental health and brain-related conditions. The DSM-5 covers a variety of mental health and brain-related conditions and disorders such as neurodevelopmental disorders (e.g., autism spectrum disorder, attention-deficit/hyperactivity disorder, learning disorder such as dyslexia and dyscalculia), schizophrenia spectrum and psychotic disorders (e.g., schizophrenia, schizoaffective disorder, delusional disorder, bipolar and related disorders, cyclothymic disorder, depressive disorder, major depressive disorder, persistent depressive disorder, anxiety disorder, social anxiety disorder, separation anxiety disorder, panic disorder, and phobias), obsessive-compulsive disorder (e.g., obsessive compulsive disorder, hoarding order, and body dysmorphic disorder), trauma and stressor related disorders (e.g., post-traumatic stress disorder and acute stress disorder), dissociative disorders (e.g., dissociative identity disorder, dissociative amnesia, and depersonalization disorder), somatic symptoms (e.g., somatic symptom disorder, illness anxiety disorder, and functional neurological symptom disorder), eating disorders (e.g., anorexia nervosa, bulimia nervosa, binge-eating disorder, and pica), elimination disorders (e.g., enuresis, sleep-wake disorder, insomnia disorder, narcolepsy, sleep apnea disorder, nightmare disorder, restless legs syndrome), sexual dysfunctions, gender dysphoria-related disorders, disruptive and impulse control disorders (e.g., oppositional defiant disorder, antisocial personality disorder, kleptomania, and pyromania), substance-related and addictive disorder (e.g., alcohol use disorder, inhalant use disorder, opioid use disorder, withdrawal-relate symptoms), neurocognitive disorders (e.g., delirium, Alzheimer's disease, Parkinson's disease, Huntington's disease, and traumatic brain injury), personality disorders (e.g., borderline personality disorder and narcissistic personality disorder), and sexual behavior disorders.

The methods disclosed herein may relate to receiving one or more wellness states. Wellness states may be received in a variety of ways. In one example, the subject may provide information relating to the subject's mental, physical, and/or social wellness states during the setup phase of a software application interfacing locally or remotely with the computer system performing the method described herein. For example, such application may ask the subject progressively more specific questions about the subject's mental, physical, and/or social wellness states based on the subject's prior responses to the questions. In another example, the subject or a third party, such as a caretaker or healthcare professional, may upload various documents related to the subject's wellness states to such application, such as prior diagnoses, prescriptions, medical records and history, medical surveys, therapy notes, and questionnaires. In another example, the application may generate prompts to obtain the subject's wellness states, such as “how are you feeling mentally today?”, “how are you feeling socially today?”, or “how are you feeling physically today?” The subject may provide written and/or verbal answers to the prompts and provide the application with information on the subject's wellness states.

### Determining a Change in Data

The methods disclosed herein may relate to determining a change in data of a first wellness type.

Determining a change in data of a first wellness type may include comparing a first data of the first wellness type against a second data of the same wellness type. For example, if the data is quantifiable, the first data may be subtracted from the second data (or vice versa) to determine the change. Examples of quantifiable physical measurements include heart rate, weight, distances, menstrual flow, blood pressure, steps taken, calories burned, sleep hours, body fat percentage, and muscle mass.

In some examples, however, qualitative data is received. Qualitative responses may be converted into quantitative data using a technique called coding. In coding, the qualitative data is broken down into discrete parts and labelled with codes. These codes can then be grouped into categories, themes, or patterns. Quantifiable information may then be generated by counting the frequency of each code, theme, or category. For example, a subject's employment status may be coded “1” for employed and “0” for unemployed. Thus, if the employment status code at one point in time is of a different value than the employment status code at a different point in time, a change in employment status data may be determined.

As another example, changes to a subject's mental data may be determined by coding a subject's qualitative mental wellness data to corresponding PHQ-9 scores. For instance, if a subject provides qualitative mental data indicating that the subject is “mildly depressed,” such data may be coded with a PHQ-9 score of 6, corresponding to “mild depression.” If the subject later provides data indicating that the subject is “severely depressed,” such data may be coded with a PHQ-9 score of to 22, corresponding to “severe depression.” The non-zero difference between 22 and 6 indicates that a change has occurred in the data of the first wellness type.

### Recommendations

The methods may relate to generating a recommendation.

In some embodiments, the recommendation is directed to an improvement of a wellness state. For example, the recommendation may be directed to an improvement of a physical wellness state, a social wellness state, a mental wellness state, or a combination thereof.

In some embodiments, the recommendation is directed to an improvement of a physical wellness state. The physical wellness states may include a physical wellness state in the “Wellness States” section herein. For example, the physical wellness state may be obesity, and the recommendation may include encouraging the subject to develop an exercise plan, consume fewer calories, enroll in an education program about diet, or the like. In some examples, the recommendation directed to an improvement of a physical wellness state may correlate with or cause an improvement to a social wellness state or a mental wellness state.

In some embodiments, the recommendation is directed to an improvement of a social wellness state. The social wellness states may include a social wellness state in the “Wellness States” section herein. For example, the social wellness state may be unemployment, and the recommendation may include encouraging the subject to enroll in a skills-based education program, see a career coach, or access resources to bolster resume and interviewing skills. In some examples, the recommendation directed to an improvement of a social wellness state may correlate with or cause an improvement to a physical wellness state or a mental wellness state.

In some embodiments, the recommendation is directed to an improvement of a mental wellness state. The mental wellness states may include a mental wellness state in the “Wellness States” section herein. For example, the mental wellness state may be depression, and the recommendation may include encouraging the subject to see a therapist or a doctor to assess medication options. In some examples, the recommendation directed to an improvement of a mental wellness state may correlate with or cause an improvement to a social wellness state or a physical wellness state.

One or more recommendations may be generated. The one or more recommendations may be generated in a variety of ways. The one or more recommendations may be of different types. For example, the one or more recommendations may be preprogrammed, user-provided, or machine learning generated.

In one example, the recommendations are preprogrammed. For example, the application may store a variety of predetermined and/or preprogrammed recommendations to improve mental, social, and physical wellness states. In one aspect, where the mental wellness state is anxiety, an example of a preprogrammed recommendation may include recommending that the subject perform breathing exercises and meditation.

In one example, the recommendations are subject-provided. For example, the application may prompt the subject to input into the application the actions (or inactions) that the subject believes would work best or have worked best for the subject to improve a mental, social, or physical wellness state (which may include those mental, social, and physical wellness states that the subject is most susceptible to). For example, a subject may input into the application that going on a walk or playing with a pet improves the subject's depression. The application may store these subject-provided actions (and inactions) e.g. and recommend them to the subject when the subject is determined to be experiencing depression.

In one example, the recommendations are generated using machine learning. For example, the application may analyze a database of recommendations that have worked for other subjects in similar situations, and provide the subject with one or more of the best-ranked recommendations.

Determining that an Action has been Taken

The methods may relate to determining that an action based on the recommendation has been taken. The action taken may include developing a plan to achieve a goal, routinely scheduling certain practices or acts in one's schedule, or seeking guidance from a professional. The action taken may include a physical action. The action taken may include no action.

In some embodiments, the action taken may be based on the recommendation. The recommendation may be a recommendation described in the “Recommendations” section herein. For example, the recommendation may be for a subject to get more sunlight exposure, and the action taken may be for the subject to allocate time each day to go outside for sunlight exposure. As another example, the recommendation may be for a subject to get more physical exercise, and the action taken may be for the subject to sign up for exercise classes that meet routinely. As yet another example, the recommendation may be for a subject to move out of an abusive living environment (e.g., living with an abusive person) and the action taken may be for the subject to find alternative housing.

In some embodiments, the action taken may be used to determine an improvement of a wellness state. For example, the action taken may be directed to improving a subject's physical wellness state, social wellness state, mental wellness state, or a combination thereof.

In some embodiments, an improvement of a wellness state of one wellness type is determined based on an improvement of a wellness state of another wellness type. For example, based on an improvement of a physical wellness state, an improvement of a social wellness state or mental wellness state is determined. As another example, based on an improvement of a social wellness state, an improvement of a mental wellness state or physical wellness state is determined. As yet another example, based on an improvement of a mental wellness state, an improvement of a physical wellness state or social wellness state is determined.

As an illustration, an improvement to a physical wellness state (e.g., tachycardia) is determined based on an improvement to a mental wellness state (e.g., from agitated to calm). In some examples, the purpose of this determination step is to confirm or deny the hypothesis that a wellness state (e.g., agitation) of a wellness type (e.g., mental) other than the wellness type being observed (e.g., physical) or the wellness type of the data (e.g., heart rate) the subject is presenting with or is being monitored or otherwise received, is the cause for the wellness state (e.g., tachycardia) of the wellness type (e.g., physical) associated with such data of that same wellness type or which such data of such same wellness type may otherwise implicate or be suggestive of (e.g., elevated resting heart rate may be associated with, implicate, or suggest tachycardia). By intentionally exploring a hypothesis in a wellness type different from the wellness type of the presenting data, the focus is forcibly shifted away from typically quick, symptomatic relief in the wellness state of the presenting wellness type, and instead to the identification of deeply rooted causes in different wellness types that manifest or masquerade as a deficient wellness state in the wellness type of the presenting data. Such hypothesis, if true, would broaden and complement the range of possible diagnoses, options, knowledge, and treatments available to a subject and a health care practitioner beyond the knee-jerk reflex of addressing the wellness state of the same wellness type as the presenting data.

### Determining a Correlation

The methods disclosed herein may relate to determining a correlation. In some embodiments, the correlation is between or among the taken action, the improvement of the wellness state of the second wellness type, and the improvement of the wellness state of the first wellness type.

The correlation may be a percentage. For example, the correlation may be a 100% correlation. The correlation may be a 5% or more correlation, a 10% or more correlation, a 15% or more correlation, a 20% or more correlation, a 25% or more correlation, a 30% or more correlation, a 35% or more correlation, a 40% or more correlation, a 45% or more correlation, a 50% or more correlation, a 55% or more correlation, a 60% or more correlation, a 65% or more correlation, a 70% or more correlation, a 75% or more correlation, a 80% or more correlation, a 85% or more correlation, a 90% or more correlation, a 95% or more correlation, or a 100% or more correlation. The correlation may be a 100% or less correlation, the correlation may be a 95% or less correlation, the correlation may be a 90% or less correlation, the correlation may be a 85% or less correlation, the correlation may be a 80% or less correlation, the correlation may be a 75% or less correlation, the correlation may be a 70% or less correlation, the correlation may be a 65% or less correlation, the correlation may be a 60% or less correlation, the correlation may be a 55% or less correlation, the correlation may be a 50% or less correlation, the correlation may be a 45% or less correlation, the correlation may be a 40% or less correlation, the correlation may be a 35% or less correlation, the correlation may be a 30% or less correlation, the correlation may be a 25% or less correlation, the correlation may be a 20% or less correlation, the correlation may be a 15% or less correlation, the correlation may be a 10% or less correlation, or the correlation may be a 5% or less correlation.

In some examples, the correlation may be calculated by taking a percentage of the number of times that the subject has performed the recommendation, reported an improvement to the second wellness state, and thereafter observed an improvement to the first wellness state, versus the number of times that the user has performed the recommendation, reported an improvement to the second wellness state, and not observed an improvement to the first wellness state.

In some examples, as described above, the strength of a correlation is measured by a correlation coefficient. In some examples, the correlation coefficient is calculated using statistical techniques such as in the Pearson correlation, Kendall rank correlation, the Spearman Correlation, the Point-Biserial correlation, the Correlation Ratio, and Cramer's V. In some examples, the correlation coefficient between or among the taken action, the improvement of the wellness state of the second wellness type, and the improvement of the wellness state of the first wellness type, is determined. In some examples, the calculation of the correlation coefficient proceeds pairwise. A first correlation coefficient may be calculated between the taken action and the improvement of the wellness state of the second wellness type. Then, a second correlation coefficient may be calculated between such improvement of the wellness state of the second wellness type to an improvement of the wellness state of the first wellness type. A third, overall coefficient indicative of the relationship between or among the taken action, the improvement to the wellness state of the second wellness type, and the improvement to the wellness state of the first wellness type, may then be calculated by multiplying or averaging the first and the second coefficients.

### Determining Causal and Non-Causal Relationships

The methods disclosed herein may relate to determining a causal relationship.

A causal relationship may be determined where a threshold measure of correlation is met or exceeded. For example, a causal relationship may be determined where a correlation of 50% or more, 55% or more, 60% or more, 65% or more, 70% or more, 75% or more, 80% or more, 85% or more, 90% or more, or 95% or more is determined between or among the taken action, the improvement of the wellness state of the second wellness type, and the improvement of the wellness state of the first wellness type. In some examples, a causal relationship may be determined where the correlation coefficient between or among the taken action, the improvement of the wellness state of the second wellness type, and the improvement of the wellness state of the first wellness type exceeds 0.5, 0.55, 0.60, 0.65, 0.70, 0.75, 0.80, 0.85, 0.90, 0.95, 0.96, 0.97, 0.98, 0.99, or more.

A causal relationship may be determined where temporal sequencing or chronological order is established. Temporal sequencing or chronological order may be established by receiving information and determining that the wellness state (e.g., PTSD) of the second wellness type (e.g., mental) precedes the first data of the first wellness type and the second data of the first wellness type, which in turn precedes the change in the wellness state of the second wellness type (e.g., onset of a traumatic PTSD episode), which in turn precedes the recommendation (e.g., breathing exercises) and the performance of the recommendation to ameliorate the wellness state of the second wellness type, which in turn precedes a determination that the performed action ameliorated the wellness state of the second wellness type (e.g., PTSD), which in turn precedes an improvement to the wellness state of the first wellness type (e.g., tachycardia). Temporal sequencing and such precession may be established based on the subject's responses to questions, the subject's medical history, and other sources of information about the subject, as well as from the actual or approximate times (including from timestamps and subject-reported times) at which such first data and such second data is received, the change in the wellness state of the second wellness type occurred, the recommendation is generated, accessed, and displayed, the action based on the recommendation was taken, the improvement to the wellness state of the second wellness type occurred, and the improvement to the wellness state of the first wellness type occurred. For example, it may be determined that an improvement in the subject's anxiety came before an improvement in the subject's heart rate. To establish temporal sequencing, the application may prompt the subject with questions such as “how long ago did you feel like you were agitated?”, “how long ago did you feel like your heart rate was elevated?”, “how long ago did you feel like the air quality deteriorated?”, or the like. The application may also gather data to establish temporal sequencing. For example, the application may gather brain wave data, which may allow the application to determine the time that the subject's mental illness began or improved. In some examples, causation is determined when there is both temporal sequencing and a correlation coefficient value that is above a minimum threshold for causation (which threshold may be higher than the minimum threshold for correlation). In some examples, such threshold value is static (e.g., r>0.9). In some examples, such threshold value is dynamic (e.g., top quintile of r values). In some examples, such threshold value is dynamic with a static floor (e.g., top quintile of r values above 0.9).

The methods disclosed herein may relate to determining a non-causal relationship. A non-causal relationship may be determined where a threshold measure of correlation is not met or exceeded. For example, a non-causal relationship may be determined where a correlation of 50% or less, 45% or less, 40% or less, 35% or less, 30% or less, 25% or less, 20% or less, 15% or less, 10% or less, or 5% or less is determined between or among the taken action, the improvement of the wellness state of the second wellness type, and the improvement of the wellness state of the first wellness type. In some examples, a non-causal relationship may be determined where the correlation coefficient between or among the taken action, the improvement of the wellness state of the second wellness type, and the improvement of the wellness state of the first wellness type does not exceed 0.5, 0.45, 0.40, 0.35, 0.30, 0.25, 0.20, 0.15, 0.10, 0.05, 0.04, 0.03, 0.02, 0.01, or less. A non-causal relationship may be determined where either temporal sequencing is not established or the value of the correlation coefficient is below a minimum threshold for causation (e.g., r<0.9).

The criteria for a non-causal relationship (e.g., lack of temporal sequencing or low correlation coefficient) may be used to determine that the change to the wellness state of the second wellness type (e.g., onset of a PTSD episode), is not caused by the change between the first data of the first wellness type and the second data of the first wellness type (e.g., increase in resting heart rate from 60 bpm to 120 bpm). For example, the change to the wellness state of the second wellness type (e.g., onset of a PTSD episode) may not be preceded by the change between the first data of the first wellness type and the second data of the first wellness type (e.g., increase in resting heart rate from 60 bpm to 120 bpm), and in fact, the opposite may be true. Further, the correlation coefficient measuring the correlation between (i) the change in the wellness state of the second wellness type (e.g., onset of a PTSD episode) and (ii) the change between the first data of the first wellness type and the second data of the first wellness type, may be below a minimum threshold for causation (e.g., r<0.9).

### Diagnosis

The methods disclosed herein may relate to suggesting a diagnosis. In some examples, the diagnosis is suggested based on the conclusion that the improvement to the wellness state of the first wellness type is caused by the improvement to the wellness state of the second wellness type, or that the change to the wellness state of the second wellness type is not caused by the change between the first data of the first wellness type and the second data of the first wellness type.

In some embodiments, the suggested diagnosis is provided to the subject. In some examples, the suggested diagnosis is provided to a healthcare professional. Non-limiting examples of healthcare professionals include clinicians, nurses, physical assistants, physical therapists, dentists, behavioral health professionals, psychologists, nutritionists, medical assistants, pharmacists, nursing assistants, pharmacy technicians, medical administrative personnel, medical interns, medical scribes, medical secretaries, or any individual associated with healthcare.

The diagnosis described herein may be a physical diagnosis, a social diagnosis, a mental diagnosis, or a combination thereof. The diagnosis may fall into one or more categories of a physical, social, and mental diagnosis. The diagnosis may not fall into any of a physical, social, and mental diagnosis.

In some embodiments, the diagnosis may be a physical diagnosis. In some embodiments, the physical diagnosis may relate to a physical injury, such as an injury to a bone, muscle, tendon, ligament, or the like.

In some embodiments, the physical diagnosis may relate to diagnosis of a disease or ailment. In some embodiments, the physical diagnoses may relate to a sexually transmitted disease (STD). Non-limiting examples of STDs include chlamydia gonorrhea, herpes (oral and genital), HIV/AIDS, hepatitis B, genital warts, hepatitis C, and trichomoniasis. In some embodiments, the physical diagnosis may relate to a respiratory disease. Non-limiting examples of respiratory diseases include asthma, chronic obstructive pulmonary disease, bronchiectasis, bronchitis, pulmonary fibrosis, sarcoidosis, and pneumonia. In some embodiments, the physical diagnosis may relate to a blood disease. Non-limiting examples of blood diseases include anemia, bleeding disorders, blood clots, and blood cancers (e.g., leukemia, lymphoma, myeloma). In some embodiments, the physical diagnosis may relate to an infection. Non-limiting examples of infections include viral infections, bacterial infections, fungal infections, parasitic infections,

In some embodiments, the physical diagnosis may relate to diagnosis of a cancer. In some embodiments, the physical diagnosis may relate to a likelihood or a suggestion of a cancer. Non-limiting examples of cancers include pancreatic cancer, prostate cancer, myeloma, thyroid cancer, breast cancer, colorectal cancer, kidney cancer, bladder cancer, brain cancer, liver cancer, lung cancer, leukemia, and stomach cancer. The cancer may be a stage of a cancer. For example, the cancer may be stage I cancer, stage II cancer, stage III cancer, or stage IV cancer. The physical diagnosis may relate to diagnosis of a tumor. The tumor may be benign or malignant.

In some embodiments, the diagnosis may be a social diagnosis. In some embodiments, the social diagnosis is loneliness. In some embodiments, the social diagnosis is neighborhood and built environment, such as a poor-quality neighborhood and built environment.

In some embodiments, the diagnosis may be a mental diagnosis. In some embodiments, the mental diagnosis is a depression disorder. In some embodiments, the mental diagnosis is an anxiety disorder. In some embodiments, the mental diagnosis is post-traumatic stress disorder (PTSD). In some embodiments, the mental diagnosis is schizophrenia. In some embodiments, the mental diagnosis is an eating disorder, such as for example, bulimia nervosa, binge eating disorder, avoidant restrictive food intake disorder, or rumination disorder. In some embodiments, the mental diagnosis is a neurodevelopmental disorder. For example, the neurodevelopmental disorder may relate to autism or attention deficit hyperactivity disorder (ADHD) In some embodiments, the mental diagnosis is a disruptive behavior or dissocial disorder.

### Treatment

The methods disclosed herein may relate to a treatment. In some embodiments, the methods relate to suggesting a treatment. In some embodiments, the methods relate to suggesting a change in a treatment.

Suggestions for such treatments or changes thereto may be provided to a healthcare professional, for example, to clinicians, nurses, physical assistants, physical therapists, dentists, behavioral health professionals, psychologists, nutritionists, medical assistants, pharmacists, nursing assistants, pharmacy technicians, medical administrative personnel, medical interns, medical scribes, medical secretaries, or any individual associated with healthcare.

The treatment may be a physical treatment, a social treatment, or a mental treatment, or a combination thereof. The treatment may fall into one or more categories of a physical, social, and mental treatment. The treatment may not fall into any of a physical, social, and mental treatment.

In some embodiments, the treatment may be a physical treatment. The physical treatment may relate to healing a bone, ligament, tendon, ligament, muscle, organ, or the like.

In some embodiments, the treatment may be a social treatment. The social treatment may relate to moving the subject to an alternative living environment.

In some embodiments, the treatment may be a mental treatment. The mental treatment may relate to the prescription of drugs. The mental treatment may relate to a therapy. For example, the therapy may be one or more of cognitive behavioral therapy, dialectical behavioral therapy, psychotherapy, art therapy, acceptance and commitment therapy, group therapy, attachment therapy, eye movement desensitization and reprocessing therapy, family systems therapy, exposure therapy, interpersonal psychotherapy, behavioral therapy, cognitive therapy, psychodynamics, integrative or holistic therapy, drug therapy, or a combination thereof.

For example, a subject may have been previously prescribed a medication, such as antidepressants, to treat the subject's mental illness. However, based on the methods herein, it may be concluded that the onset of depression (as presented by the change between the first data of the first wellness type and the second data of the first wellness type) is temporally preceded by and strongly correlated with a change to the subject's social and community context (second wellness state) from a loving and accepting social environment to a discriminatory one. Thus, a change to the subject's treatment may be suggested to instead or in addition improve or address the change to the second wellness state (e.g., social and community context).

### Determining a Wellness Score

The methods disclosed herein may relate to determining a wellness score for a wellness type of a subject. The wellness score can correspond to physical wellness, mental wellness, or social wellness. In some embodiments, the methods may relate to determining a plurality of wellness scores to a plurality of wellness types.

In some embodiments, the wellness score comprises a numerical value. A magnitude of the numerical value can be proportional and/or inversely proportional to a wellness level. For example, a greater numerical value can correspond to a higher wellness level and a lower numerical value can correspond to a lower wellness level. Alternatively, a greater numerical value can correspond to a lower wellness level and a lower numerical value can correspond to a higher wellness level. A range of the numerical value can be about 0 to about 10, about 0 to about 20, about 0 to about 30, about 0 to about 50, about 0 to about 100, about 0 to about 200, about 0 to about 300, about 0 to about 500, or about 0 to about 1000. In some embodiments, the wellness score comprises a categorical value. The categorical value can correspond to a category of wellness. For example, the categorical value can correspond to high wellness, moderate wellness, or low wellness.

In some embodiments, the methods can comprise generating an aggregate wellness score. The aggregate wellness score can be determined based on aggregating individual wellness scores determined for two or more wellness types. The aggregate wellness score can be a sum, an average, a median, or a mode of the individual wellness scores. A range of the numerical value can be about 0 to about 10, about 0 to about 20, about 0 to about 30, about 0 to about 50, about 0 to about 100, about 0 to about 200, about 0 to about 300, about 0 to about 500, about 0 to about 1000, about 0 to about 2000, or about 0 to about 3000.

In some embodiments, the wellness score is determined based at least in part on clinical data associated with the subject. The clinical data can comprise one or more of: biometrics data, clinical notes, subject input, and medical codes. The biometrics data can comprise a heart rate, a breathing rate, a blood pressure, a blood oxygen level, a blood sugar, a blood cholesterol, a temperature, a height, and/or a weight. The clinical notes can comprise medical records, medical professional notes, prescriptions, and/or social worker notes. The subject input can comprise answers to questions from a medical professional, answer to questions from a social worker, a description of a physical symptom, a description of an emotional state, and/or a description of a social state. The subject input can be provided by the subject, the medical professional, the social worker, and/or a family member of the subject. The medical codes can comprise electronic medical record codes and/or insurance codes used to diagnose, describe, or classify the subject's wellness state.

In some embodiments, the method comprises extracting a plurality of clinical features from the clinical data. The plurality of clinical features can comprise clinical diagnoses, data associated with the wellness type, data associated with another wellness type, a medical history, and/or demographic data. Extracting the plurality of clinical features can comprise performing a statistical analysis of the input data. For example, the input data can comprise heart rate, and extracting the plurality of clinical features can comprise determining an average heart rate. Extracting the plurality of clinical features can comprise summarizing the input data. For example, the input data can comprise a plurality of medical codes and extracting the plurality of clinical features can comprise summarizing the codes associated with the wellness type. Extracting the plurality of clinical features can comprise determining a physical, mental, and/or social wellness state based on the input data. For example, the input data can comprise a plurality of self-reported emotional states and extracting the plurality of clinical features can comprise determining whether the subject suffers from depression or anxiety.

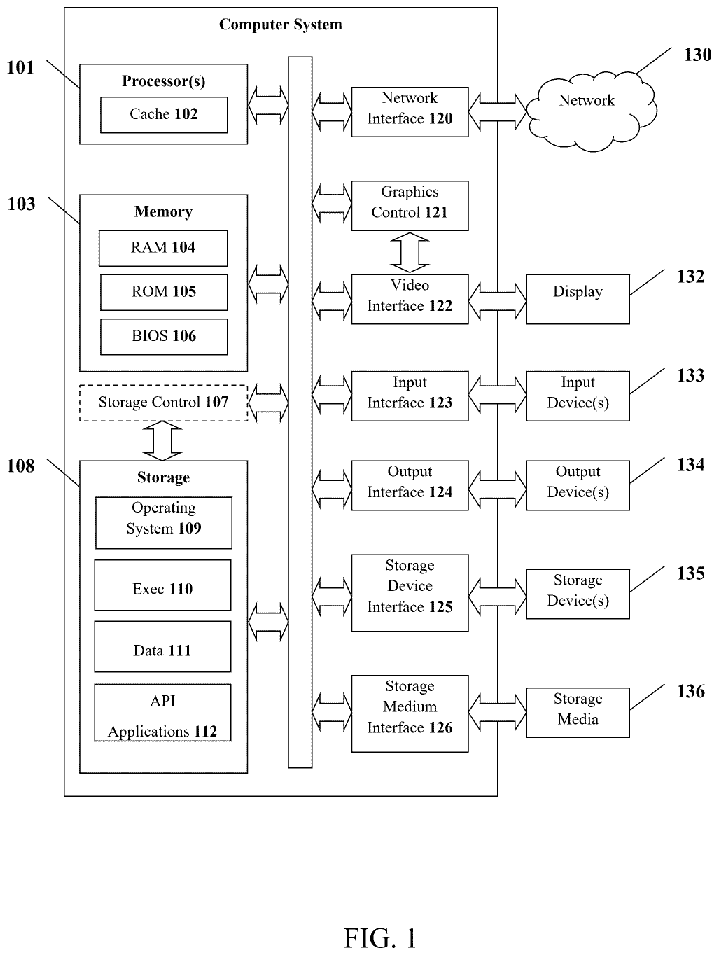

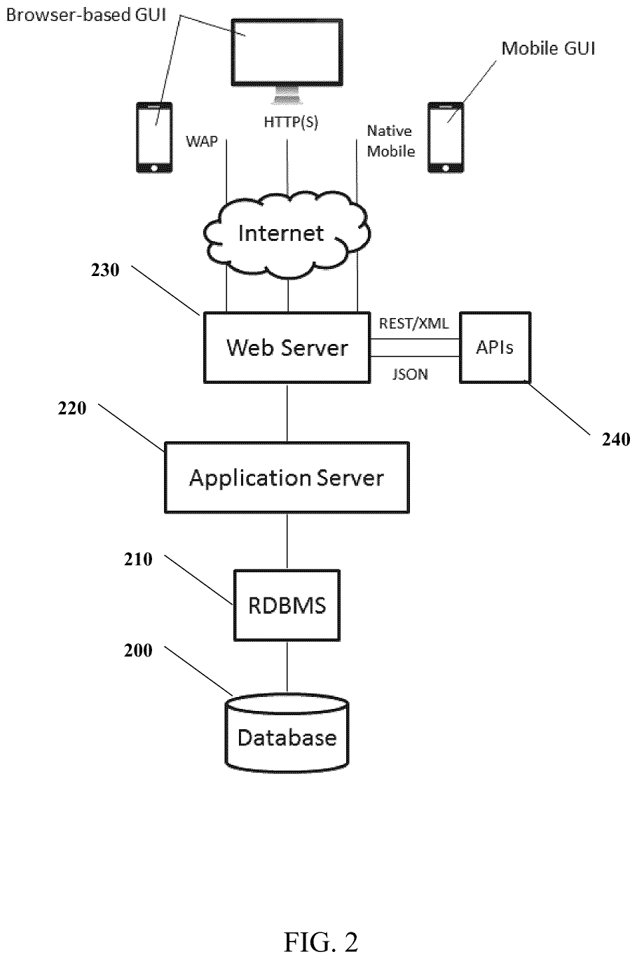

In some embodiments, the clinical data comprises text (e.g., clinical notes, text-based subject input, etc.). In some embodiments, extracting the plurality of clinical features comprises processing the clinical data with a large language model (LLM). The processing with the LLM can be performed by computer system 100 depicted in FIG. 1, application server 220 depicted in FIG. 2, or web server 230. Non-transitory, processor-executable instructions for the processing with the LLM can be stored at memory 103, storage 108, storage devices 135, storage medium 136, and/or database 200. The processing with the LLM can be executed by processor(s) 101. The text can be provided as input to the LLM. The LLM can process the text to determine key features associated with the wellness type. The LLM can be trained on clinical notes. The LLM can be pre-trained on general texts and fine-tuned on clinical notes. Processing the text can comprise training and/or updating the LLM. The LLM can identify patterns or correlations within the text. The LLM can identify portions of the text that are relevant to the wellness type.

In some embodiments, the wellness score is determined by an algorithm. The algorithm can be configured to output the wellness score based on performing one or more operations on the plurality of clinical features. The one or more operations can comprise: pre-processing, transformation, featurization, dimensionality reduction, machine learning, and/or statistical analysis. The algorithm can comprise a machine learning algorithm trained to predict a wellness score based on the plurality of clinical features. The algorithm can comprise a mathematical algorithm configured to perform a set of mathematical operations on the plurality of clinical features to generate the wellness score.

In some embodiments, the algorithm determines the wellness score based at least in part on a plurality of weights. In some embodiments, each of the plurality of weights can be associated with a corresponding clinical feature. The plurality of weights can be indicative of a level of contribution of the corresponding clinical feature to the wellness score. A subset of clinical features having a high level of contribution to the wellness score can be determined based on the subset corresponding to weights having a high magnitude. The method can comprise comparing each of the weights to a predetermined threshold to determine the subset of clinical features having the high level of contribution to the wellness score.

In some embodiments, the method comprises training the machine learning algorithm. Training the machine learning algorithm can comprise obtaining training data comprising input data and associated ground truth wellness scores. Training the machine learning algorithm can comprise updating the plurality of weights to minimize a difference between predicted wellness scores and the ground truth wellness scores.

In some embodiments, the wellness score is outputted. In some embodiments, the aggregate wellness score is outputted. The wellness score or the aggregate wellness score can be outputted to a medical professional, the subject, the social worker, and/or the family member. The outputted wellness or aggregate wellness score can be considered when determining a diagnosis, a treatment plan, or suggested actions. The outputted wellness or aggregate wellness score can be considered when evaluating an overall health of the subject. The methods can comprise assigning and outputting a plurality of wellness scores or aggregate wellness scores corresponding to a plurality of time points. The plurality of wellness scores or aggregate wellness scores can be plotted to determine a wellness trajectory of the subject.

In some embodiments, the subset of clinical features having the high level of contribution to the wellness score is outputted. The subset of clinical features can be outputted to the medical professional, the subject, the social worker, and/or the family member. The subset of clinical features can be outputted along with the wellness score or the aggregate wellness score in a clinical report. The subset of clinical features can help the medical professional, the subject, the social worker, and/or the family member determine key clinical features contributing to a high or a low wellness score.

In some embodiments, the method comprises generating a recommendation based at least in part on the wellness score, the aggregate wellness score, and/or the subset of clinical features having the high level of contribution to the wellness score. Generating the recommendation can comprise determining if an action is necessary, based on the wellness score. For example, if the wellness score is within a normal range, a recommendation may not be generated. Generating the recommendation can comprise determining a severity of the wellness score. For example, if a wellness score is moderate, the recommendation can comprise lifestyle changes (e.g., diet, exercise), and if the wellness score is more severe, the recommendation can comprise medical intervention (e.g., medication, surgery). Generating the recommendation can comprise evaluating the subset of clinical features having the high level of contribution. For example, if it is determined that a mental issue has a high level of contribution to a low physical wellness score, the recommendation can comprise actions aimed at resolving the mental issue.

### EXAMPLES

### Physical Data and Social Wellness State

In one aspect, the methods relate to data of a physical wellness type and a wellness state of a social wellness type. For example, the physical wellness data may relate to a subject's breathing and respiration rate (e.g., sleeping breathing rate), and the social wellness state may relate to the subject's neighborhood and built environment (e.g., neighborhood air quality).

The methods may comprise receiving a first breathing rate physical data and a second breathing rate physical data. The first physical data received may be the subject's sleeping breathing rate at a first time point, and the second physical data received may be the subject's sleeping breathing rate at a second time point. The first physical data received may be that the subject has a normal sleeping breathing rate, such as 15 breaths per minute, and the second physical data received may be that the subject has a high sleeping breathing rate, such as 30 breaths per minute.

The methods may include receiving a wellness state of a second wellness type. A social wellness state (e.g., neighborhood air quality) may be received. The social wellness state may be received in any way. In one example, the subject or a third party may provide their address as part of initializing the application. In another example, the subject's address may be included in a medical form uploaded to the application by the subject or a third party, such as a healthcare professional, or a caretaker. In another example, the subject or a third party may provide an indication of air quality at the subject's address. In yet another example, the application may use the subject's address (e.g., learned through the means above) to periodically send a query to an air quality information service through a network such as the internet and receive periodic air quality data corresponding to the air quality at or near the subject's address.

A change in the social wellness type (e.g., neighborhood air quality) may be determined based on the change between the first physical data and the second physical data (e.g., elevated sleeping breathing rate). For example, the elevated sleeping breathing rate of the subject may trigger a collection and analysis of available information relating to the subject's neighborhood to determine the cause of the elevated breathing rate of the subject. Such neighborhood data may indicate a deterioration in the air quality in the subject's neighborhood near the subject's address. Thus, in some examples, the determination of the change in the wellness state of the second wellness type (e.g., neighborhood air quality) may be in response to and therefore based on the change between the first physical data and the second physical data (e.g., increased breathing rate).

A recommendation may be generated based on the change in the subject's social state (e.g., neighborhood air quality), and the recommendation may be directed to improving such social state (e.g., neighborhood air quality). One or more recommendations may be generated. For example, one recommendation may be encouraging the subject to move to a neighborhood or built environment with better air quality. Another example may be to recommend that the subject install air filters and/or air purifiers in the home. Another example of a recommendation may be to recommend to the subject to keep windows closed in the home, utilize exhaust fans in the home, and use appropriate air conditioning circulation modes in the home (e.g., recirculation vs. open ventilation). Another example may be to recommend that the subject put on a particulate filtration mask or similar air filtration mask when outdoors or exposed to polluted air.

The methods may comprise receiving information and determining that an action based on the recommendation has been taken. In one example, the subject may input information into the application. For example, the subject may perform an action based on the generated recommendations, such as keeping the windows closed in the home and wearing a filtration mask. The subject then may indicate an improvement or worsening in health into the application, indicating whether the action took place. Based on the subject's response, the application may determine that the actions based on the recommendations (e.g., closing the windows and wearing a filtration mask) have been taken.

The methods may comprise determining an improvement of the social wellness state (e.g., neighborhood air quality) based on the action taken. For example, the subject may take the action of closing the windows in the home and wearing a filtration mask. The closure of the windows may reduce the harmful particulate count in the subject's living environment, and such count may be periodically provided to the application by a particulate sensor. Based on the reduction in the harmful particulate count after the recommendation is performed, an improvement to the social wellness state may be determined. In some examples, the subject may indicate an improvement or increase in the subject's social wellness state (e.g., neighborhood air quality) in the application based on these actions. Thus, the application may determine that the subject's social wellness state (e.g., neighborhood air quality) was improved based on the subject's actions of closing the windows in the home and wearing a filtration mask.

The methods may comprise determining an improvement of the physical wellness state (e.g., sleeping breathing rate) based on the improvement of the social wellness state (e.g., neighborhood air quality). After the subject has acted on the recommendation (e.g., closed the windows in the home and wear a filtration mask), the subject's sleeping breathing rate may be measured to determine if, as a result of such action and improvement to the subject's air quality, there has been an improvement to the sleeping breathing rate as compared to the initially received data indicating an elevated sleeping breathing rate. In one aspect, the subject manually enters information on the subject's sleeping breathing rate, for example, based on a recording of the subject's breathing while the subject was sleeping. In another aspect, a sensor, such as a watch or a ring that collects data about the subject while the subject is sleeping, periodically transmits or provides sleeping breathing rate data to the application. For example, it may be determined that the subject's sleeping breathing rate went down (to a normal level) after the action was performed and the subject's air quality improved, and as such, the application may make a determination of an improvement in the subject's physical wellness state (e.g., sleeping breathing rate) based on the improvement on the subject's social wellness state (e.g., access to clean air in the neighborhood).

The method may comprise determining a correlation between or among the taken action, the improvement in the subject's social wellness state (e.g., access to clean air in the neighborhood), and the improvement of the subject's physical wellness state (e.g., sleeping breathing rate). For example, the relationship between or among such action, improvement in the subject's social wellness state, and improvement to the subject's physical wellness state, may be measured using a correlation coefficient. If there is temporal sequencing and the correlation coefficient is above a minimum threshold (e.g., r>0.7 or a correlation percentage of greater than 70%), then it may be determined that a correlation exists between or among the taken action, the improvement in the subject's social wellness state, and the improvement in the subject's physical wellness state.

The methods may include concluding a causal or non-causal relationship between the improvement to the physical wellness state (e.g., sleeping breathing rate) and the improvement to the social wellness state (e.g., access to clean air in the neighborhood). For example, it may be concluded based on the presence of temporal sequencing and a high degree of correlation (e.g., r>0.9 or a correlation percentage of greater than 90%) between or among the taken action, the improvement to the subject's social wellness state, and the improvement to the subject's physical wellness state, that the action taken caused the improvement to the subject's social wellness state (e.g., access to clean air in the neighborhood) which caused the improvement to the subject's physical wellness state (e.g., normal sleeping breathing rate). As another example, it may be concluded based on the presence of temporal sequencing and a high degree of correlation (e.g., r>0.9 or a correlation percentage of greater than 90%) between the changes to the subject's social wellness state and the changes to (i) the subject's physical wellness state before the change in the subject's social wellness state (e.g., before the deterioration in air quality) and before the action was taken; (ii) the subject's physical wellness state after the change in the subject's social wellness state (e.g., after the deterioration in air quality) and before the action was taken; and/or (iii) the subject's physical wellness state after the improvement to the subject's social wellness state (e.g., after obtaining access to clean air) as a result of the action taken, that the change in the subject's social wellness state (e.g., a lack of access to clean air in the neighborhood) caused the change in the subject's physical wellness state (e.g., increase in sleeping breathing rate). Further, for example, it may be concluded that the change in the subject's social state (e.g., deterioration in air quality) is not caused by the change in the subject's physical wellness state (e.g., increase in sleeping breathing rate). Such non-causal relationship may be determined based on the lack of temporal sequencing or a low degree of correlation (e.g., r<0.1 or a correlation percentage of less than 10%) between or among the changes to the subject's social wellness state and the changes to (i) the subject's physical wellness state before the change in the subject's social wellness state (e.g., before the deterioration in air quality) and before the action was taken; (ii) the subject's physical wellness state after the change in the subject's social wellness state (e.g., after the deterioration in air quality) and before the action was taken; and/or (iii) the subject's physical wellness state after the improvement to the subject's social wellness state (e.g., after obtaining access to clean air) as a result of the action taken. In some examples therefore, it may be concluded that there is no causal relationship between the subject's social wellness state and the subject's physical wellness state, or that the change in the subject's physical wellness state is not the cause for the change in the subject's social wellness state.

The methods may include suggesting a diagnosis with respect to the social wellness state. For example, the application, using a machine learning technique described herein, may receive as inputs, and may process, information relating to the subject's physical wellness and social wellness, including physical and social wellness data and physical and social wellness states before and after the performance of the recommended action, and information about the action performed, to generate an output in the form of a suggested diagnosis. For example, based on the conclusion that the subject's abnormally high sleeping breathing rate (i.e., the change in the physical wellness state) is caused by the subject's lack of access to clean air in the neighborhood (i.e., the change in the social wellness state), the application may suggest that the diagnosis for the subject's high sleeping breathing rate is insufficient access to clean air in the neighborhood (i.e., a social rather than physical wellness issue).

The methods may include suggesting a change to a treatment with respect to the physical wellness state or the social wellness state. For example, the subject may have been previously prescribed a medication, such as benzodiazepines, to treat the subject's high sleeping breathing rate. However, based on the suggested diagnosis that the high sleeping breathing rate is caused by the lack of access to clean air in the neighborhood, a change to such treatment may be suggested, such that the suggested treatment is instead or additionally directed to improving the subject's social wellness state, such as increasing the subject's access to clean air in the subject's neighborhood. For example, one suggestion may be to install air filters and purifiers in the home. Another suggestion may be to close the windows in the home to keep out airborne contaminants.

### Physical Data and Mental Wellness State

In one aspect, the methods relate to data of a physical wellness type and a wellness state of a mental wellness type. For example, the physical wellness data may relate to a subject's heart or pulse rate (e.g., resting heart rate), and the mental wellness state may relate to the subject's mental health (e.g., anxiety).

The methods may comprise receiving a first resting heart rate physical data and a second resting heart rate physical data. The first physical data received may be the subject's resting heart rate a first time point, and the second physical data received may be the subject's resting heart rate at a second time point. The first physical data received may be that the subject has a normal resting heart rate, such as 70 beats per minute (bpm), and the second physical data received may be that the subject has a high resting heart rate, such as 110 bpm.

The methods may include receiving a wellness state of a second wellness type. A mental wellness state (e.g., anxiety) may be received. The mental wellness state may be received in any way. In one example, the subject or a third party may provide their medical records, including mental health medical records, to the application during application setup. In another example, the subject or a third party may provide a record of the subject's anxiety levels over a period of time into the application. In another example, the subject or a third party may provide the subject's therapist's notes during therapy sessions with the subject into the application. In yet another example, a device, such as a watch, ring, or implant that collects data about the subject may periodically transmit or provide the subject's anxiety levels (or proxies therefor, such as the level of cortisol or other anxiety-related hormones in the subject's fluids) over a period of time to the application. In some examples, the subject is periodically asked questions about how the subject feels, or to rate the subject's anxiety levels, particularly when it hasn't been the subject's day, week, month, or even year. In some examples, the subject may respond with adjectives or verbs describing how the subject is feeling. In some examples, the subject's ratings may be numerical and a lower number may be associated with a lower level of anxiety, and a higher number may be associated with a higher level of anxiety.

A change in the mental wellness type (e.g., anxiety) may be determined based on the change between the first physical data and the second physical data (e.g., elevated heart rate). For example, the elevated heart rate of the subject may trigger a collection and analysis of available information relating to the subject to determine the cause of the elevated heart rate of the subject. Such information may include measurements of the subject's anxiety levels (or proxies therefor, such as the level of cortisol or other anxiety-related hormones in the subject's fluids), or the subject's responses to questions about how the subject is feeling or to rate the subject's anxiety, which may be used, alone or in combination, to determine that the subject's state of anxiety has increased (e.g., worsened).

A recommendation may be generated based on the change in the subject's mental state (e.g., calm to anxious), and the recommendation may be directed to improving such mental state (e.g., anxiety). One or more recommendations may be generated. For example, one recommendation may be encouraging the subject to enroll in therapy, see a psychiatrist to explore medication options, or both. Another example of a recommendation may be to recommend that the subject practice deep breathing exercises. Additional examples of recommendations may be to recommend the subject to develop a fitness routine, spend more time outdoors, and practice meditation.

The methods may comprise receiving information and determining that an action based on the recommendation has been taken. In one example, the determination may be made based on information input by or for the subject into the application. For example, the subject may perform an action based on the generated recommendations, such as practicing meditation and deep breathing exercises. The subject may then input information into the application to indicate that the recommended action was performed. In some examples, the subject may also indicate whether the action improved or worsened the subject's mental wellness state (e.g., anxiety). Thus, the application may determine that the actions based on the recommendations generated (e.g., practicing meditation and deep breathing exercises) have been taken.

The methods may comprise determining an improvement of the mental wellness state (e.g., anxiety) based on the actions taken. In some examples, the improvement is determined without subject input. In some examples, the improvement is determined with subject input. For example, the subject may take the action of practicing meditation and deep breathing exercises. Thereafter, the subject's anxiety levels (or proxies therefor) may be measured to determine if, as a result of such action, there has been an improvement to the subject's anxiety levels (or proxies therefor) as compared to the initially received anxiety data (or proxies therefor). In some examples, the meditation and deep breathing exercises may reduce the subject's measured anxiety levels (or proxies therefor, such as the level of cortisol or other anxiety-related hormones in the subject's fluids). Based on the reduction in such measurements after the recommendation is performed, an improvement to the mental wellness state may be determined.

In some examples, the subject may provide feedback to indicate that the subject's mental wellness state was improved by performing the recommended actions. For example, in one aspect, the subject may be asked to rate the impact of the performance of the recommended action on the subject's mental wellness state based on a scoring system in which a score of 100 indicates maximum improvement and a score of −100 indicates maximum deterioration. Thus, if the subject assigns a positive score to the taken recommended action (e.g., practicing meditation and deep breathing exercises), it may be determined that the subject's mental wellness state (e.g., anxiety) was improved based on such taken actions.

The methods may comprise determining an improvement of the physical wellness state (e.g., resting heart rate) based on the improvement of the mental wellness state (e.g., anxiety). After the subject has acted on the recommendation (e.g., practiced meditation and deep breathing exercises), the subject may input information into the application indicating a decrease in anxiety. In one aspect, the subject manually enters information on the subject's level of anxiety, such as information on the subject's emotional state (e.g., happy, contented, sad, angry). In another aspect, a sensor, such as a watch or a ring connected to the subject transmits or provides anxiety information (e.g., measurements of anxiety or proxies therefor) to the application after the subject has performed the recommended action. Such anxiety information may indicate that the subject's level of anxiety decreased after performing the action. The receipt of such input from the subject (indicating a decrease in anxiety) or such information from a sensor, may trigger the collection and analysis of the subject's heart rate to determine if, as a result of such action and improvement to the subject's anxiety, the subject's heart rate has also improved as compared to the initially received data indicating a high heart rate. For example, it may be determined that the subject's resting heart rate went down (to a normal level) after the action was performed and the subject's anxiety improved, and as such, a determination of an improvement in the subject's physical state (e.g., resting heart rate) may be made based on the improvement on the subject's mental state (e.g., anxiety).

The method may comprise determining a correlation between or among the taken action, the improvement in the subject's mental wellness state (e.g., anxiety), and the improvement in the subject's physical wellness state (e.g., resting heart rate). For example, the relationship between or among such action, improvement in the subject's mental wellness state, and improvement to the subject's physical wellness state, may be measured using a correlation coefficient. If there is temporal sequencing and the correlation coefficient is above a minimum threshold (e.g., r>0.7 or a correlation percentage of greater than 70%), then a correlation may be determined between or among the subject's action (e.g., practicing meditation and deep breathing exercises), the improvement of the subject's mental wellness state (e.g., anxiety), and the improvement of the subject's physical wellness state (e.g., resting heart rate).

The methods may include concluding a causal or non-causal relationship between the improvement to the mental wellness state (e.g., anxiety) and the improvement to the physical wellness state (e.g., resting heart rate). For example, it may be concluded based on the presence of temporal sequencing and a high degree of correlation (e.g., r>0.9 or a correlation percentage of greater than 90%) between or among the taken action, the improvement to the subject's mental wellness state, and the improvement to the subject's physical wellness state, that the action taken caused the improvement to the subject's mental wellness state (e.g., reduced levels of anxiety) which caused the improvement to the subject's physical state (e.g., reduced resting heart rate). As another example, it may be concluded based on the presence of temporal sequencing and a high degree of correlation (e.g., r>0.9) between or among the changes in the subject's mental wellness state and changes in (i) the subject's physical wellness state before the change in the subject's mental wellness state (e.g., before the onset of an anxiety attack) and before the action was taken; (ii) the subject's physical wellness state after the change in the subject's mental wellness state (e.g., after the onset of an anxiety attack) and before the action was taken; and/or (iii) the subject's physical wellness state after the improvement to the subject's mental wellness state (e.g., reduction of anxiety levels) as a result of the action taken, that the change in the subject's mental wellness state (e.g., onset of an anxiety attack) caused the change in the subject's physical wellness state (e.g., high resting heart rate). Further, for example, it may be concluded that the change in the subject's mental wellness state (e.g., onset of an anxiety attack) is not caused by the change in the subject's physical wellness state (e.g., high resting heart rate). Such non-causal relationship may be determined based on the absence of temporal sequencing or a low degree of correlation (e.g., r<0.1 or a correlation percentage of less than 10%) between or among the changes to the subject's mental wellness state and changes to (i) the subject's physical wellness state before the change in the subject's mental wellness state (e.g., before the onset of an anxiety attack) and before the action was taken; (ii) the subject's physical wellness state after the change in the subject's mental wellness state (e.g., after the onset of an anxiety attack) and before the action was taken; and/or (iii) the subject's physical wellness state after the improvement to the subject's mental wellness state (e.g., reduction in anxiety levels) as a result of the action taken. In some examples therefore, it may be concluded that there is no causal relationship between the subject's mental wellness state and physical wellness state, or that the change in the subject's physical wellness state is not the cause for the change in the subject's mental wellness state.

The methods may include suggesting a diagnosis with respect to the mental wellness state. For example, the application, using a machine learning technique described herein, may receive as inputs, and may process, information relating to the subject's physical wellness and mental wellness, including physical and mental wellness data and physical and mental wellness states before and after the performance of the recommended action, and information about the action performed, to generate an output in the form of a suggested diagnosis. In one aspect, the application may suggest a diagnosis to the mental wellness state (e.g., anxiety). For example, based on the conclusion that the subject's elevated resting heart rate (i.e., physical wellness state) is caused by an increase in the subject's anxiety levels (i.e., mental wellness state), the application may suggest that the diagnosis for the subject's high resting heart rate is high anxiety levels (i.e., a mental rather than physical wellness issue), and therefore mental in nature. Thus, the application may suggest that the subject has a type of anxiety disorder, such as a panic disorder, a social anxiety disorder, or separation anxiety disorder, rather than tachycardia.

The methods may include suggesting a change to a treatment with respect to the physical wellness state or the mental wellness state. For example, the subject may have been previously prescribed a medication, such as beta blockers, to treat the subject's high resting heart rate. However, based on the suggested diagnosis of an anxiety disorder, a change to such treatment may be suggested, such that the suggested treatment is instead or additionally directed to improving the subject's mental wellness state, such as meditation, deep breathing, or psychotherapy to decrease the subject's anxiety levels.

### Mental Data and Social Wellness State

In one aspect, the methods relate to data of a mental wellness type and a wellness state of a social wellness type. For example, the mental wellness data may relate to a subject mental health (e.g., anxiety) measured by a GAD-7 scale. The GAD-7 scale is a questionnaire that asks a subject to rate the severity of their anxiety symptoms over a period of time. The GAD-7 scale provides the subject with a total score, ranging from 0 to 21, where 0-4 would be considered having minimal anxiety; 5-9 would be considered having mild anxiety; 10-14 would be considered having moderate anxiety; and 15-21 would be considered as having severe anxiety. The social wellness state may relate to the subject's social and community environment (e.g., persons that the subject lives with). For example, the social wellness type may relate to whether the subject lives with an abusive person or not, for example, an abusive family member, spouse, boyfriend, girlfriend, or roommate.

The methods may comprise receiving a first anxiety mental data and a second anxiety mental data. The first mental data received may be the subject's anxiety levels at a first time point, and the second mental data received may be the subject's anxiety levels at a second time point. The first mental data received may be that the subject has a minimal level of anxiety (e.g., total score ranging from 0-4 according to the GAD-7 scale), and the second mental data received may be that the subject has a high level of anxiety (e.g., total score ranging from 15-21 according to the GAD-7 scale).

The methods may include receiving a wellness state of a second wellness type. A social wellness state (e.g., persons that the subject lives with) may be received. The social wellness state may be received in various ways. In one example, the subject or a third party may provide their living information, such as who the subject lives with and their relationship to that person, during application setup. In another example, the subject or a third party may indicate the people that the subject lives with, as well as the subject's feelings towards those people, in the application. In yet another example, the subject or a third party may provide to the application the subject's address and the application may query and receive periodic updates on the people that lives at the subject's address based on available census data. The application may also query any police reports have been filed by a person living at the subject's address.

A change in the social wellness state (e.g., persons that the subject lives with) may be determined based on the change between the first mental data and the second mental data (e.g., elevated levels of anxiety). For example, the elevated anxiety levels of the subject may trigger a collection and analysis of available information relating to the subject's social state (e.g. persons that the subject lives with) to determine the cause of the elevated anxiety levels of the subject. Such social data may indicate an increase of abusiveness of the subject's roommates. Thus, in some examples, the determination of the change in the wellness state of the second wellness type (e.g., social state) may be in response to and therefore based on the change between the first mental data and the second mental data (e.g., elevated levels of anxiety).

A recommendation may be generated based on the change in the subject's social state (e.g., persons the subject lives with), and the recommendation may be directed to improving such social state (e.g., persons the subject lives with). One or more recommendations may be generated. For example, one recommendation may be encouraging the subject to pursue alternative housing arrangements, such as affordable housing opportunities. Another example of a recommendation may be to recommend that the subject engage in relationship counseling with the subject's abusive roommate. Another recommendation may be recommending that the subject install locks on the subject's bedroom door to avoid contact with the abusive person. Another example of a recommendation may be to encourage the subject to move in with persons that the subject has a healthy relationship with, such as a parent, friend, or sibling.

The methods may comprise receiving information and determining that an action based on the recommendation has been taken. In one example, the subject may input information into the application. For example, the user may perform an action based on the generated recommendations, such as finding alternative housing arrangements. In some examples, the application may periodically receive the subject's location based on a location sensor, such as based on global positioning satellite (GPS) data and determine that the subject has moved away from the prior address and therefore performed the action of finding alternative housing arrangements. Based on the subject's response or such sensor data, the application may determine that the actions based on the recommendations (e.g., finding alternative housing arrangements) have been taken.

The methods may comprise determining an improvement of the social wellness state (e.g., persons the subject lives with) based on the action taken. In some examples, the improvement is determined without subject input. In some examples, the improvement is determined with subject input. For example, after the subject has taken the recommended action of finding alternative housing arrangements, the subject may be asked to rate the impact of the performance of the recommended action on the subject's mental wellness state based on a scoring system in which a score of 100 indicates maximum improvement and a score of −100 indicates maximum deterioration. Thus, if the subject assigns a positive score to the taken recommended action (e.g., finding alternative housing arrangements), it may be determined that the subject's social wellness state (e.g., persons the subject lives with) was improved based on such taken actions.

The methods may comprise determining an improvement of the mental wellness state (e.g., anxiety) based on the improvement of the social wellness state (e.g., persons the subject lives with). After the subject has acted on the recommendation (e.g., found alternative housing arrangements), and after the application has received information that the subject has done so (e.g., the subject has provided a rating for the action), the subject may be promptly asked to input information into the application relating to his or her anxiety level. In one aspect, the subject manually enters information into the application on the subject's anxiety levels. For example, the subject may take a test that provides a level of anxiety on the GAD-7 scale. The subject may manually enter their results that indicate an improvement in the subject's anxiety into the application. In another aspect, a sensor, such as a watch or a ring collecting data about the subject provides information relating to a measure of the subject's anxiety level (or proxies therefor) to the application. If the subject's levels of anxiety went down, it may be determined that the improvement in the subject's mental wellness state (e.g., anxiety) was based on the improvement in the subject's social wellness state (e.g., the subject living with non-abusive people).

The methods may comprise determining a correlation between or among the action taken, the improvement in the subject's social wellness state (e.g., persons the subject lives with), and the improvement of the subject's mental wellness state (e.g., anxiety). For example, the relationship between or among such action, improvement in the subject's social wellness state, and improvement to the subject's mental wellness state, may be measured using a correlation coefficient. If there is temporal sequencing and the correlation coefficient is above a minimum threshold (e.g., r>0.7 or a correlation percentage of greater than 70%), then a correlation may be determined between or among the subject's action (e.g., finding alternative housing), the improvement of the subject's social wellness state (e.g., living with a non-abusive person), and the improvement of the subject's mental wellness state (e.g., decreased levels of anxiety).

The methods may include concluding a causal or non-causal relationship between the improvement to the mental wellness state (e.g., anxiety) and the improvement to the social wellness state (e.g., home environment). For example, it may be concluded based on the presence of temporal sequencing and a high degree of correlation (e.g., r>0.9 or a correlation percentage of greater than 90%) between or among the taken action, the improvement to the subject's social wellness state, and the improvement to the subject's mental wellness state, that the action taken caused the improvement to the subject's social wellness state (e.g., living with a non-abusive person) which caused the improvement to the subject's mental wellness state (e.g., decreased levels of anxiety). As another example, it may be concluded that the improvement in the subject's social wellness state (e.g., not living with an abusive person) caused the improvement in the subject's mental wellness state (e.g., no or low levels of anxiety). Further, for example, if there is temporal sequencing and a high degree of correlation (e.g., r>0.9 or a correlation percentage of over 90%) between or among the changes to the subject's social wellness state and changes to (i) the subject's mental wellness state before the change in the subject's social wellness state (e.g., before living with abusive persons) and before the action was taken; (ii) the subject's mental wellness state after the change in the subject's social wellness state (e.g., after living with abusive persons) and before the action was taken; and/or (iii) the subject's mental wellness state after the improvement to the subject's social wellness state (e.g., after not living with abusive persons) as a result of the action taken, it may be concluded that the change in the subject's social state (e.g., living with an abusive person) caused the change in the subject's mental wellness state (e.g., anxiety). Further, for example, it may be concluded that the change in the subject's social wellness state (e.g., living with abusive persons) is not caused by the change in the subject's mental wellness state (e.g., anxiety). Such non-causal relationship may be determined based on the absence of temporal sequencing or a low degree of correlation (e.g., r<0.1 or a correlation percentage of less than 10%) between or among changes in the subject's social wellness state and changes in (i) the subject's mental wellness state before the change in the subject's social wellness state (e.g., before living with abusive persons) and before the action was taken; (ii) the subject's mental wellness state after the change in the subject's social wellness state (e.g., after living with abusive persons) and before the action was taken; and/or (iii) the subject's mental wellness state after the improvement to the subject's social wellness state (e.g., after not living with non-abusive persons) as a result of the action taken. In some examples therefore, it may be concluded that there is no causal relationship between the subject's social wellness state and physical wellness state, or that the change in the subject's mental wellness state is not the cause for the change in the subject's social wellness state.

The methods may include suggesting a diagnosis with respect to the social wellness state. For example, the application, using a machine learning technique described herein, may receive as inputs, and may process, information relating to the subject's mental wellness and social wellness, including mental and social wellness data and mental and social wellness states before and after the performance of the recommended action, and information about the action performed, to generate an output in the form of a suggested diagnosis. For example, based on the conclusion that the subject's anxiety (i.e., the change in the subject's mental wellness state) is caused by the subject's abusive home environment (i.e., the change in the subject's social wellness state), the application may suggest that the diagnosis for the subject's anxiety is an abusive home environment (i.e., a social rather than mental wellness issue).

The methods may include suggesting a change to a treatment with respect to the mental wellness state or the social wellness state. For example, the subject may have been previously prescribed a medication, such as benzodiazepines, to treat the subject's anxiety. However, based on the suggested diagnosis that the subject's anxiety is caused by an abusive home environment, a change to such treatment may be suggested, such that the suggested treatment is instead or additionally directed to improving the subject's social wellness state, such as moving to an abuse-free home environment. For example, one suggestion may be to find alternative housing away from the abusive person. Another suggestion may be to engage in relationship therapy with the abusive person to stop the abuse.

### Mental Data and Physical Wellness State

In one aspect, the methods relate to data of a mental wellness type and a wellness state of a physical type. For example, the mental wellness data may relate to a subject's mental health (e.g., level of depression). The subject's level of depression may be measured by a PHQ-9. The PHQ-9 may assess the severity of a subject's depressive symptoms over a period of time with total scores ranging from 0-27, where a score of 0-4 would be considered minimal depression or no depression; a score of 5-9 would be considered mild depression severity; a score of 10-14 would be considered moderate depression severity; a score of 15-19 would be considered moderately-severe depression; and a score of 20-27 would be considered severe depression. The physical wellness state may relate the subject's unhealthy weight gain or obesity.

The methods may comprise receiving a first depression mental data and a second depression mental data. The first mental data received may be the subject's level of depression at a first time point, and the second mental data received may be the subject's level of depression at a second time point. The first mental data received may be that the subject is not depressed or has minimal depression (e.g., a total score of 0-4 according to the PHQ-9), and the second mental data received may be that the subject has high or severe depression (e.g., a total score of 20-27 according to the PHQ-9).

The methods may include receiving a wellness state of a second wellness type. A physical wellness state (e.g., obesity/weight gain) may be received. The physical wellness state may be received in any way. In one example, the subject or a third party may provide their history of weight gain during application set-up. In another example, the subject's medical history, including the subject's weight history, may be provided to the application by the subject or a third party, such as a healthcare professional, or a caretaker. In yet another example, the application may periodically query or receive periodic weight data relating to the subject from a sensor that collects data about the subject. For example, the application may alert the subject to step on a smart scale to provide weight data to the application.

A change in the physical wellness type (e.g., obesity or weight gain) may be determined based on the change between the first mental data and the second mental data (e.g., increase in depression). For example, the increase in depression of the subject, may trigger a collection and analysis of available information relating to the subject to determine the cause of the increased depression of the subject. Such information may include the subject's weight or BMI (or proxies therefor, such as measurements of the subject's waistline), or the subject's responses to questions about whether the subject knows or feels that the subject is putting on weight, from which it may be (alone or in combination with other factors) determined that the subject has gained weight or is obese.

A recommendation may be generated based on the change in the subject's physical state (e.g., weight gain or obesity), and the recommendation may be directed to improving such physical state (e.g., weight gain or obesity). One or more recommendations may be generated. For example, one recommendation may be encouraging the subject to develop and enact a healthy eating plan. Another recommendation may be encouraging the subject to connect with a personal trainer to develop and enact an exercise routine. Another example of a recommendation may be to recommend that the subject meet with a dietitian, explore healthy food options, and establish and achieve health goals relating to diet and exercise.

The methods may comprise receiving information and determining that an action has been taken based on the recommendation. In one example, the determination may be made based on information input by or for the subject into the application. For example, the subject may perform an action based on the generated recommendations, such as developing and enacting an exercise and diet plan. The subject then may input information into the application to indicate that the recommended action was performed. In some examples, the subject may also indicate whether the action improved or worsened the subject's physical wellness state (e.g., unhealthy weight gain or obesity). Thus, the application may determine that the actions (e.g., developing and enacting an exercise and diet plan) have been taken based on the recommendations.

The methods may comprise determining an improvement of the physical wellness state (e.g., weight gain or obesity) based on the action taken. In some examples, the improvement is determined without subject input. In some examples, the improvement is determined with subject input. For example, the subject may take the action of developing an exercise and diet plan. Thereafter, the subject's weight or BMI (or proxies therefor) may be measured and the results manually input into the application by or for the subject or transmitted by smart scales to the application to determine if, as a result of developing and enacting such exercise and diet plan, there has been an improvement to the subject's weight or BMI (or proxies therefor) as compared to the initially received weight or BMI data (or proxies therefor). In some examples, the enactment of the exercise and diet plan may reduce the subject's weight or BMI (or proxies therefor, such as the subject's waistline). Based on the reduction in such measurements after the recommendation is performed, an improvement to the physical wellness state (e.g., unhealthy weight gain or obesity) may be determined.

In some examples, the subject may provide feedback to indicate that the subject's physical wellness state was improved by performing the recommended actions. For example, the subject may be asked to rate the impact of the performance of the recommended action on the subject's physical wellness state based on a scoring system in which a score of 100 indicates maximum improvement and a score of −100 indicates maximum deterioration. Thus, if the subject assigns a positive score to the taken recommended action (e.g., developing and enacting an exercise and diet plan), it may be determined that the subject's physical wellness state (e.g., weight gain or obesity) was improved based on the subject's actions of developing and enacting an exercise and diet plan.

The methods may comprise determining an improvement of the mental wellness state (e.g., depression) based on the improvement of the physical wellness state (e.g., weight gain or obesity). After the subject has acted on the recommendation (e.g., to develop and enact an exercise and diet plan) and after the application has received information that the subject has done so (e.g., the subject has provided a rating for the action), the subject may be promptly asked to input information into the application relating to his or her depression levels. In one aspect, the subject manually enters information on the subject's depression. For example, the subject may take a test to indicate the subject's level of depression according to the PHQ-9 score and input such score into the application. In another aspect, a sensor, such as a watch, ring, or implant collecting data about the subject provides information relating to a measure of the subject's depression level (or proxies therefor, such as the levels of certain mood-regulating neurotransmitters in the subject's fluids) to the application. If the subject's levels of depression went down after the subject lost weight and became non-obese as a result of the recommended action, it may be determined that the improvement in the subject's mental wellness state (e.g., depression) was based on the improvement on the subject's physical wellness state (e.g., weight gain/obesity).

The method may comprise determining a correlation between or among the taken action, the improvement in the subject's physical wellness state (e.g., weight gain/obesity), and the improvement of the subject's mental wellness state (e.g., depression). For example, the relationship between or among such action, improvement in the subject's physical wellness state, and improvement to the subject's mental wellness state, may be measured using a correlation coefficient. If there is temporal sequencing and the correlation coefficient is above a minimum threshold (e.g., r>0.7 or a correlation percentage of greater than 70%), then a correlation may be determined between or among the subject's action (e.g., developing and implementing an exercise and diet plan), the improvement of the subject's physical state (e.g., weight gain/obesity), and the improvement of the subject's mental wellness state (e.g., depression).

The methods may include concluding a causal or non-causal relationship between the improvement to the mental wellness state (e.g., depression) and the improvement to the physical wellness state (e.g., weight gain/obesity). For example, it may be concluded based on the presence of temporal sequencing and a high degree of correlation (e.g., r>0.9 or a correlation percentage of greater than 90%) between or among the taken action, the improvement to the subject's physical wellness state, and the improvement to the subject's mental wellness state, that the action taken caused the improvement to the subject's physical wellness state (e.g., decreased unhealthy weight gain or BMI) which caused the improvement to the subject's mental wellness state (e.g., decreased level of depression). As another example, it may be concluded that the improvements in the subject's physical wellness state (e.g., developing and implementing an exercise plan) caused the improvements in the subject's mental wellness state (e.g., decreased level of depression). Further, for example, if there is temporal sequencing and a high degree of correlation (e.g., r>0.9 or a correlation percentage of over 90%) between or among the changes in the subject's physical wellness state and the changes in (i) the subject's mental wellness state before the change in the subject's physical wellness state (e.g., before unhealthy weight gain or obesity) and before the action was taken; (ii) the subject's mental wellness state after the change in the subject's physical wellness state (e.g., after unhealthy weight gain or obesity) and before the action was taken; and/or (iii) the subject's mental wellness state after the improvement to the subject's physical wellness state (e.g., after decreased weight gain or BMI) as a result of the action taken, it may be concluded that the change in the subject's physical state (e.g., weight gain or obesity) is not caused by the change in the subject's mental wellness state (e.g., depression). Further, for example, it may be concluded that the change in the subject's mental wellness state (e.g., onset of depression) is not caused by the change in the subject's physical wellness state (e.g., unhealthy weight gain or obesity). Such non-causal relationship may be determined based on the lack of temporal sequencing or a low degree of correlation (e.g., r<0.1 or a correlation percentage of less than 10%) between or among the changes in the subject's physical wellness state and the changes in (i) the subject's mental wellness state before the change in the subject's physical wellness state (e.g., before unhealthy weight gain or obesity) and before the action was taken; (ii) the subject's mental wellness state after the change in the subject's physical wellness state (e.g., after unhealthy weight gain or obesity) and before the action was taken; and/or (iii) the subject's mental wellness state after the improvement to the subject's physical wellness state (e.g., after decreased weight gain or BMI) as a result of the action taken. In some examples therefore, it may be concluded that there is no causal relationship between the subject's physical wellness state and mental wellness state, or that the change in the subject's mental wellness state is not the cause for the change in the subject's physical wellness state.

The methods may include suggesting a diagnosis with respect to the physical wellness state. For example, the application, using a machine learning technique described herein, may receive as inputs, and may process, information relating to the subject's physical wellness and mental wellness, including physical and mental wellness data and physical and mental wellness states before and after the performance of the recommended action, and information about the action performed, to generate an output in the form of a suggested diagnosis. For example, based on the conclusion that the subject's depression (i.e., the change in the subject's physical wellness state) is caused by the subject's unhealthy weight gain or obesity (i.e., the change in the subject's physical wellness state), the application may suggest that the diagnosis for the subject's depression is unhealthy weight gain or obesity (i.e., a physical rather than mental wellness issue).

The methods may include suggesting a change to a treatment with respect to the mental wellness state or the physical wellness state. For example, the subject may have been previously prescribed a medication, such as antidepressants, to treat the subject's increasing severity of depression. However, based on the suggested diagnosis that the subject's depression is caused by unhealthy weight gain or obesity, a change to such treatment may be suggested, such that the suggested treatment is instead or additionally directed to improving the subject's physical wellness state, such as by developing and enacting an exercise and diet plan. For example, one suggestion may be to exercise at least thirty minutes a day. Another suggestion may be to limit caloric intake to a more moderate number of calories per day.

### Social Data and Mental Wellness State

The methods may relate to receiving data of a first social wellness type and a wellness state of a mental type. For example, the social wellness data may relate to a subject's employment status (e.g., employed or unemployed), and the mental wellness state may relate to a subject's depressive state (e.g., depressed or not depressed) according to a PHQ-9 Questionnaire. The PHQ-9 Questionnaire may assess the severity of a subject's depressive symptoms over a period of time with total scores ranging from 0-27, in which a score of 0-4 would be considered minimal or no depression severity; a score of 5-9 would be considered mild depression severity; a score of 10-14 would be considered moderate depression severity; a score of 15-19 would be considered moderately-severe depression; and a score of 20-27 would be considered severe depression.

The methods may comprise receiving a first employment status social wellness data and a second employment status social wellness data. The first social wellness data may be that the subject is employed. The second social wellness data may be that the subject is unemployed.

The methods may include receiving a wellness state of a second wellness type. A mental wellness state (e.g., depression) may be received. The mental wellness state may be received in any way. In one example, the subject or a third party may self-report their depressive or mood levels into the application during setup. In another example, the subject or a third party may complete a psychological assessment scoring their depressive symptoms such as the PHQ-9 Questionnaire. The subject's score may be inputted by the subject or a third party into the application.

A change in the mental wellness type (e.g., depression) may be determined based on the change between the first social data and the second social data (e.g., employment status). For example, a change in the subject's status from employed to unemployed, may trigger a collection and analysis of available information relating to the subject to determine the cause of the change in the employment status of the subject. Such information may include the subject's depression levels (or proxies therefor, such as the levels of certain mood-regulating neurotransmitters in the subject's fluids) and the subject's PHQ-9 Questionnaire score, from which it may be (alone or in combination with other factors) determined that the subject is depressed.

A recommendation may be generated based on the determined change in the subject's mental state (e.g., depression), and the recommendation may be directed to improving such mental state (e.g., depression). One or more recommendations may be generated. For example, one recommendation may be to recommend that the subject start some form of mental therapy (e.g., cognitive behavioral therapy, interpersonal therapy, psychodynamic therapy), see a doctor to discuss prescribed medication options, or both. Another example of a recommendation may be to recommend that the subject utilize mental health resources for depressed persons, and to practice healthy habits like daily walks and exercise that can lessen depressive symptoms.

The methods may comprise receiving information and determining that an action has been taken based on the recommendation. In one example, the determination may be made based on subject information input by or for the subject into the application. For example, the subject may perform an action based on the generated recommendations, such as starting cognitive behavioral therapy with a therapist and being prescribed/taking medications. The subject then may input information into the application to indicate that the recommended action was performed. In some examples, the subject may also indicate whether the action improved or worsened the subject's mental wellness state (e.g., depression). Thus, the application may determine that the actions (e.g., starting therapy and taking medication) have been taken based on the recommendations.

The methods may comprise determining an improvement of the mental wellness state (e.g., depression) based on the action taken. In some examples, the improvement is determined without subject input. In some examples, the improvement is determined with subject input. For example, the subject may take the action of starting therapy and taking prescribed medication. Thereafter, the subject's depression (or proxies therefor) may be measured and the results manually input into the application by the user to the application to determine if, as a result of starting therapy and taking prescribed medication, there has been an improvement to the subject's depression (or proxies therefor) as compared to the initially received depression data (or proxies therefor). In some examples, the therapy and medication may reduce the subject's depression (or proxies therefor). Based on the reduction in such measurements after the recommendation is performed, an improvement to the mental wellness state may be determined.

In some examples, the subject may provide feedback to indicate that the subject's mental wellness state was improved by performing the recommended actions. For example, the subject may be asked to rate the impact of the performance of the recommended action on the subject's physical wellness state based on a scoring system in which a score of 100 indicates maximum improvement and a score of −100 indicates maximum deterioration. Thus, if the subject assigns a positive score to the taken recommended action (e.g., starting therapy and taking medication), it may be determined that the subject's mental wellness state (e.g., depression) was alleviated based on the subject's actions of starting therapy and taking prescribed medication.

The methods may comprise determining an improvement of the social wellness state (e.g., unemployment) based on the improvement of the mental wellness state (e.g., depression). After the subject has acted on the recommendation (e.g., starting therapy and taking prescribed medication) and after the application has received information that the subject has done so (e.g., the subject has provided a rating for the action), the subject may be promptly asked to input information into the application relating to the subject's employment status. In one aspect, the subject manually enters information on the subject's employment status. In another aspect, the subject's employment information is obtained from other sources, such as the subject's social media information. For example, the subject may input or it may be determined that after therapy and medication, the subject is now employed. As such, a determination of an improvement in the subject's social state (e.g., employment status) may be made based on the improvement on the subject's mental (e.g., depression).

The method may comprise determining a correlation between or among the action taken, the improvement in the subject's mental wellness state (e.g., depression), and the improvement of the subject's social wellness state (e.g., unemployment status). For example, the relationship between or among such action, improvement in the subject's mental wellness state, and improvement to the subject's social wellness state, may be measured using a correlation coefficient. If there is temporal sequencing and the correlation coefficient is above a minimum threshold (e.g., r>0.7 or a correlation percentage of greater than 70%), then a correlation may be determined between or among the subject's action (e.g., starting therapy and taking prescribed medication), the improvement of the subject's mental wellness state (e.g., depression), and the improvement of the subject's social wellness state (e.g., unemployment status).

The methods may include concluding a causal or non-causal relationship between the improvement to the social wellness state (e.g., unemployment status) and the improvement to the mental wellness state (e.g., depression). For example, it may be concluded based on the presence of temporal sequencing and a high degree of correlation (e.g., r>0.9 or a correlation percentage of greater than 90%) between or among the taken action, the improvement to the subject's mental wellness state, and the improvement to the subject's social wellness state, that the action taken caused the improvement to the subject's mental wellness state (e.g., overcoming depression) which caused the improvement to the subject's social wellness state (e.g., becoming employed). As another example, it may be concluded that the improvement in the subject's mental wellness state (e.g., overcoming depression) caused the improvement in the subject's social wellness state (e.g., gaining employment). Further, for example, if there is temporal sequencing and a high degree of correlation (e.g., r>0.9 or a correlation percentage of over 90%) between or among the changes in the subject's mental wellness state and the changes in the (i) the subject's social wellness state before the change in the subject's mental wellness state (e.g., before onset of depression) and before the action was taken; (ii) the subject's social wellness state after the change in the subject's mental wellness state (e.g., after onset of depression) and before the action was taken; and/or (iii) the subject's social wellness state after the improvement to the subject's mental wellness state (e.g., after overcoming depression) as a result of the action taken, it may be concluded that the change in the subject's mental wellness state (e.g., depression) is not caused by the change in the social wellness state (e.g., becoming unemployed). Further, for example, it may be concluded that the change in the subject's mental wellness state (e.g., onset of depression) is not caused by the change in the subject's social wellness state (e.g., unemployment). Such non-causal relationship may be determined based on the lack of temporal sequencing or a low degree of correlation (e.g., r<0.1 or a correlation percentage of less than 10%) between or among the changes in the subject's mental wellness state and the changes in the (i) the subject's social wellness state before the change in the subject's mental wellness state (e.g., before onset of depression) and before the action was taken; (ii) the subject's social wellness state after the change in the subject's mental wellness state (e.g., after onset of depression) and before the action was taken; and/or (iii) the subject's social wellness state after the improvement to the subject's mental wellness state (e.g., after overcoming depression) as a result of the action taken. In some examples therefore, it may be concluded that there is no causal relationship between the subject's mental wellness state and social wellness state, or that the change in the subject's mental wellness state is not the cause for the change in the subject's physical wellness state.

The methods may include suggesting a diagnosis with respect to the mental wellness state. For example, the application, using a machine learning technique described herein, may receive as inputs, and may process, information relating to the subject's mental wellness and social wellness, including mental and social wellness data and mental and social wellness states before and after the performance of the recommended action, and information about the action performed, to generate an output in the form of a suggested diagnosis. For example, based on the conclusion that the subject's unemployment (i.e., the change in the subject's social wellness state) is caused by the subject's depression (i.e., the change in the subject's mental wellness state), the application may suggest that the diagnosis for the subject's unemployment is depression (i.e., a mental rather than social wellness issue).

The methods may include suggesting a change to a treatment with respect to the social wellness state or the mental wellness state. For example, the subject may have previously been encouraged to seek job resources, such as talking to a recruiter or applying to a large number of jobs a day to overcome unemployment. However, based on the suggested diagnosis that the subject's unemployment is caused by depression, a change to such treatment may be suggested, such that the suggested treatment is instead or additionally directed to improving the subject's mental state. For example, a suggested treatment may be for the subject to enroll in therapy or discuss medications with a doctor to alleviate depressive symptoms.

### Social Data and Physical Wellness State

In one aspect, the methods relate to receiving data of a first social wellness type and a wellness state of a physical type. For example, the social wellness data may relate to a subject's access to quality health care (e.g., ability to obtain affordable, quality health insurance), and the physical wellness state may relate to a subject's body mass index and tobacco use.

The methods may comprise receiving a first food security social wellness data and a second food security social wellness data. The first social wellness data received may be the subject's health insurance category and premiums at a first point in time (e.g., silver tier, $1000 a month), and the second social wellness data received may be the subject's health insurance category and premiums at a second point in time (e.g., silver tier, $2000 a month). Health insurance plans may be categorized into tiers, such as a bronze tier, silver tier, gold tier, or platinum tier (correlating with a low tier, a middle tier, a high tier, and a highest tier). Generally, the higher the tier, the better the health insurance benefits but higher the premiums.

The methods may include receiving a wellness state of a second wellness type. A physical wellness state (e.g., tobacco use and BMI) may be received. The physical wellness state may be received in any way. In one example, the subject or a third party may have their tobacco use or BMI checked during a physical exam at their doctor's office and subsequently input their results in the application. In another example, the subject may monitor his or her BMI by frequently weighing himself or herself using a traditional scale or a smart scale and entering or transmitting such weight information to the application. The subject's BMI may be calculated by the application based on the user's height and weight, or the BMI may be calculated by the user or smart scale and entered or provided to the application. The subject may also enter their tobacco use information into the application in response to a question prompt. In yet another example, the subject may input their results from blood or urine tests and diagnostic imaging results (e.g., nicotine test, a chest X-ray, echocardiography, myocardial perfusion imaging via nuclear scintigraphy, magnetic resonance imaging (MRI), and computed tomography (CT), dual x-ray absorptiometry (DEXA)) into the application.

A change in the physical wellness type (e.g., heavy tobacco use and increase in BMI to unhealthy range) may be determined based on the change between the first social wellness data and the second social wellness data (e.g., substantial increase in health insurance premiums at the same tier). For example, the change in health insurance premiums of the subject may trigger a collection and analysis of available information relating to the subject to determine the cause of the change in the health insurance premiums of the subject. Such information may include the subject's tobacco use and BMI status (or proxies therefor, such as nicotine tests and diagnostic imaging results, heart rate, heart rate variability, resting heart rate, walking heart rate average, cardiovascular recovery time after exercise, cardiovascular fitness, maximal oxygen consumption (VO2 max), body fat percentage), which may be used (alone or in combination) to determine that the subject may be heavily using tobacco or has a high BMI.

A recommendation may be generated based on the change in the subject's physical wellness state (e.g., heavy increase in tobacco use and increase in BMI to unhealthy range), and the recommendation may be directed to improving the physical wellness state (e.g., reduction of tobacco use and reduction in BMI to healthy range). One or more recommendations may be generated. For example, one recommendation may be encouraging the subject to join a smoking cessation program, use nicotine replacements, consult a doctor regarding quit-smoking pills, hire a quit smoking coach, use quit smoking resources, or undertake an exercise plan or start working out with a personal trainer.

The methods may comprise receiving information and determining that an action has been taken based on the recommendation. In one example, the determination may be made based on information input into the application by or for the subject. For example, the subject may perform an action based on the generated recommendations, such as utilizing quit-smoking pills, starting an exercise plan, and beginning to work out with a personal trainer. The subject then may input information into the application to indicate that the recommended action was performed. In some examples, the subject may also indicate whether the action improved or worsened the subject's physical wellness state (e.g., tobacco use and BMI). Thus, the application may determine that the actions (e.g., quit-smoking pills and exercise) have been taken based on the recommendations.

The methods may comprise determining an improvement of the physical wellness state (e.g., diabetes and/or heart disease) based on the action taken. In some examples, the improvement is determined without subject input. In some examples, the improvement is determined with subject input. For example, the subject may take the actions of using quit-smoking pills or starting an exercise plan. Thereafter, the subject's tobacco use and BMI status (or proxies therefor) may be measured and the results manually input into the application by or for the subject or transmitted by devices such as a smart scale to the application to determine if, as a result of such use of quit-smoking pills and exercise, there has been an improvement to the subject's tobacco use and BMI status (or proxies therefor) as compared to the initially received tobacco use and BMI status (or proxies therefor). In some examples, after using the quit-smoking pills and exercising regularly, the subject quits smoking and the subject's BMI improves from the unhealthy to the healthy range. Thus, an improvement to the subject's physical wellness state (e.g., tobacco cessation and BMI reduction from unhealthy to healthy range) may be determined based on the taking of the recommended action (e.g., consult a doctor regarding quit-smoking pills and undertake an exercise plan).

In some examples, the subject may provide feedback to indicate that the subject's physical wellness state (e.g., tobacco use and BMI) was improved by performing the recommended actions. For example, the subject may be asked to rate the impact of the performance of the recommended action on the subject's physical wellness state based on a scoring system in which a score of 100 indicates maximum improvement and a score of −100 indicates maximum deterioration. Thus, if the subject assigns a positive score to the taken recommended action (e.g., consult a doctor regarding quit-smoking pills and undertake an exercise plan), it may be determined that the subject's physical wellness state (e.g., tobacco use and BMI) was improved based on the subject's actions of using quit-smoking pills and regular exercise.

The methods may comprise determining an improvement of the social wellness state (e.g., ability to obtain affordable, quality health insurance) based on the improvement of the physical wellness state (e.g., tobacco cessation and BMI reduction from unhealthy to healthy range). After the subject has acted on the recommendation (e.g., by using quit-smoking pills and exercising) and after the application has received information that the subject has done so (e.g., the subject has provided a rating for the action), the subject may be promptly asked to input information into the application relating to his or her social wellness state (e.g., whether there has been an improvement in the subject's ability to obtain affordable, quality health insurance). In one aspect, the subject searches for and manually enters updated health plan category and premium information into the application. In another aspect, the application queries such health plan category and premium information based on the subject's updated tobacco use and BMI information. The application may compare the prior health plan category and premium information with the updated health plan category and premium information to determine that after the actions of quitting smoking and exercising taken in response to the recommendation and determine, for example, that the subject's health insurance premiums or quotations therefor with the same provider or at the same tier have been substantially reduced (e.g., from $2000 per month to $750 per month for silver tier), and the subject may even upgrade to a higher tier plan for the same expenditure in premiums (e.g., $1500 per month for gold tier). Thus, a determination of an improvement in the subject's social state (e.g., ability to obtain affordable, quality health insurance) may be made based on the improvement on the subject's physical state (e.g., tobacco cessation and reduction of BMI from unhealthy to healthy range).

The method may comprise determining a correlation between or among the taken action, the improvement in the subject's physical wellness state (e.g., tobacco cessation and reduction in BMI from unhealthy to healthy range), and the improvement of the subject's social wellness state (e.g., ability to upgrade to a higher tier insurance plan without additional expenditure). For example, the relationship between or among such action, improvement in the subject's physical wellness state, and improvement to the subject's social wellness state, may be measured using a correlation coefficient. If there is temporal sequencing and the correlation coefficient is above a minimum threshold (e.g., r>0.7 or a correlation percentage of greater than 70%), then a correlation may be determined between or among the subject's action (e.g., using quit-smoking pills and exercising), the improvement of the subject's physical wellness state (e.g., tobacco cessation and reduction in BMI from unhealthy to healthy range), and the improvement of the subject's social wellness state (e.g., ability to upgrade to a higher tier health insurance plan without additional expenditure).

The methods may include concluding a causal or non-causal relationship between the improvement to the physical wellness state (e.g., smoking cessation and reduction in BMI from unhealthy to healthy range) and the improvement to the social wellness state (e.g., ability to upgrade to a higher tier health insurance plan without additional expenditure). For example, it may be concluded based on the presence of temporal sequencing and a high degree of correlation (e.g., r>0.9 or a correlation percentage of greater than 90%) between or among the taken action, the improvement to the subject's physical wellness state, and the improvement to the subject's social wellness state, that the action taken caused the improvement to the subject's physical wellness state (e.g., tobacco cessation and BMI reduction from unhealthy to healthy range) which caused the subject's social wellness state (e.g., ability to upgrade to a higher tier health insurance plan without additional expenditure). As another example, it may be concluded that the improvement to the subject's physical wellness state (e.g., smoking cessation and BMI reduction from unhealthy to healthy range) caused the improvement to the subject's social wellness state (e.g., food secure). Further, for example, if there is temporal sequencing and a high degree of correlation (e.g., r>0.9 or a correlation percentage of over 90%) between or among the changes in the subject's physical wellness state and the changes in the (i) the subject's social wellness state before the change in the subject's physical wellness state (e.g., before smoking cessation and BMI reduction) and before the action was taken; (ii) the subject's social wellness state after the change in the subject's physical wellness state (e.g., after smoking cessation and BMI reduction) and before the action was taken; and/or (iii) the subject's social wellness state after the improvement to the subject's physical wellness state (e.g., after smoking cessation and BMI reduction) as a result of the action taken, it may be concluded that the change in the subject's physical state (e.g., smoking cessation and BMI reduction) is not caused by the change in the social wellness state (e.g., ability to upgrade to a higher tier health insurance plan without additional expenditure).

In some examples, it may be concluded that there is no causal relationship between the subject's physical wellness state and social wellness state. Such non-causal relationship may be determined based on the lack of temporal sequencing or a low degree of correlation (e.g., r<0.1 or a correlation percentage of less than 10%) between or among the changes in the subject's physical wellness state and the changes in the (i) the subject's social wellness state before the change in the subject's physical wellness state (e.g., before smoking cessation and BMI reduction) and before the action was taken; (ii) the subject's social wellness state after the change in the subject's physical wellness state (e.g., after smoking cessation and BMI reduction) and before the action was taken; and/or (iii) the subject's social wellness state after the improvement to the subject's physical wellness state (e.g., after smoking cessation and BMI reduction) as a result of the action taken.

The methods may include suggesting a diagnosis with respect to the physical wellness state. For example, the application, using a machine learning technique described herein, may receive as inputs, and may process, information relating to the subject's physical wellness and social wellness, including physical and social wellness data and physical and social wellness states before and after the performance of the recommended action, and information about the action performed, to generate an output in the form of a suggested diagnosis. For example, based on the conclusion that the subject's ability to procure higher tier health insurance without additional expenditure (i.e., the change in the subject's social wellness state) is caused by the subject's smoking cessation and BMI reduction (i.e., the change in the subject's physical wellness state), the application may suggest that the diagnosis for the subject's increase in insurance premiums is the subject's use of tobacco and unhealthy BMI (i.e., a physical rather than social wellness issue).

The methods may include suggesting a change to a treatment with respect to the social wellness state or the physical wellness state. For example, the subject may have previously been focused on directly improving their social wellness state by searching for lower-priced health plans or reducing their benefits. However, based on the suggested diagnosis, a change to such treatment may be suggested, such that the suggested treatment is instead or additionally directed to improving the subject's physical wellness state, such as managing the subject's tobacco use and BMI. For example, one suggestion may be to sustain the subject's smoking cessation by participating in sustained care smoking cessation programs. Another suggestion may be to continue to engage in exercise but also consider a healthy diet plan to stabilize the BMI improvements.

### Aggregate Wellness Score

In one aspect, the methods relate to generating an aggregate wellness score based on a first wellness type and a second wellness type for a subject. For example, the first wellness type and the second wellness type can correspond to physical wellness and social wellness. The first wellness type and the second wellness type can correspond to physical wellness and mental wellness. The first wellness type and the second wellness type can correspond to social wellness and mental wellness. In some embodiments, the methods relate to generating an aggregate wellness score based on the first wellness type, the second wellness type, and a third wellness type. The first wellness type, the second wellness type, and the third wellness type can correspond to physical wellness, social wellness, and mental wellness.

The method can comprise obtaining clinical data associated with the subject. For example, the method can comprise accessing a medical history of the subject to obtain clinical notes and medical codes provided by a medical professional during one or more appointments with the subject. The method can comprise obtaining clinical notes taken by a social worker during one or more sessions with the subject. The method can comprise obtaining a daily blood pressure from the subject. The method can comprise obtaining daily inputs from the subject about any physical, mental, or social states they experienced that day (e.g., a pain level, a mood, and social interactions).

The method can comprise processing the clinical data to extract a plurality of clinical features. The plurality of clinical features can correspond to any physical symptoms, mental states, or social conditions the subject experiences. For example, extracting the plurality of clinical features can comprise determining that the subject has high blood pressure, has a history of heart disease, has chronic pain, experiences anxiety, and lacks a social support system. Extracting the plurality of clinical features can comprise performing statistical analyses, for example, calculating an average blood pressure for the subject and determining that it is greater than a threshold value. Extracting the plurality of clinical features can comprise processing text with an LLM. The LLM can be applied to the medical history, the social worker notes, and the daily inputs to extract the clinical features. For example, the LLM can determine that the subject has a history of heart disease from the medical history, determine that the subject has chronic pain and experiences anxiety from the daily inputs, and determine that the subject lack a social support system based on the social worker notes. The LLM can be previously trained on medical histories, social worker notes, and/or subject inputs to extract relevant clinical features.

The method can comprise determining the first wellness score using a first wellness algorithm. The first wellness algorithm can be a machine learning algorithm that takes as input the clinical features and outputs the first wellness score. For example, the first wellness score can be a physical wellness score, and the first wellness algorithm can be a physical wellness algorithm. The machine learning algorithm can determine the first wellness score based on a plurality of weights associated with each of the clinical features. A magnitude of each of the weights can indicate a level of contribution of the corresponding clinical feature to the wellness score. For example, the chronic pain may have a higher level of contribution to the physical wellness score compared to the anxiety, so a weight associated with the chronic pain can have a higher magnitude than the anxiety. The machine learning algorithm can be previously trained using training data. The training data can comprise sets of clinical features and associated ground truth first wellness scores, and the machine learning algorithm can be trained to minimize a difference between determined first wellness scores and the ground truth first wellness scores. The training can comprise updating the weights associated with each of the clinical features. The first wellness score can comprise a numerical value proportional to a level of wellness of the first wellness type. For example, the first wellness score can be a physical wellness score ranging from 1 to 300, and a higher wellness score can correspond to a higher level of physical wellness.

The method can comprise determining the second wellness score using a second wellness algorithm. The second wellness algorithm can be the same or a different algorithm as the first wellness algorithm. The first wellness algorithm can be a same type of algorithm as the second wellness algorithm, but have different weights associated with each of the clinical features. For example, the first wellness algorithm can be a physical wellness algorithm and the second wellness algorithm can be a social wellness algorithm, and lack of social support systems may have a lower associated weight for the physical wellness algorithm and a higher associated weight for the social wellness algorithm. The second wellness algorithm can be trained based on training data comprising sets of clinical features and associated ground truth second wellness scores. The second wellness score can comprise a numerical value proportional to a level of wellness of the second wellness type. For example, the second wellness score can be a social wellness score ranging from 1 to 300, and a higher score can correspond to a higher level of social wellness.

The method can comprise determining the aggregate wellness score based on the first and the second wellness scores. The aggregate wellness score can be determined based on a physical wellness score and a social wellness score. The aggregate wellness score can be determined based on a physical wellness score and a mental wellness score. The aggregate wellness score can be determined based on a social wellness score and a mental wellness score. In some embodiments, the aggregate wellness score can be determined based on the physical wellness score, social wellness score, and the mental wellness score. Determining the aggregate wellness score can comprise adding the first and the second wellness scores. Determining the aggregate wellness score can comprise determining an average of the first and the second wellness scores. A numerical value of the aggregate wellness score can correspond to a level of overall wellness. For example, the aggregate wellness score can range from 2 to 600, and a higher value can correspond to a higher level of overall wellness.

The method can comprise outputting the first wellness score, the second wellness score, and/or the aggregate wellness score. The method can comprise determining a subset of clinical features having a highest level of contribution to the first wellness score, the second wellness score and/or the aggregate wellness score, The subset of clinical features can be determined based on evaluating the weights of the first wellness algorithm and the second wellness algorithm. The subset of clinical features can be outputted along with the wellness scores to help a medical professional, a social worker, the subject, and/or a family member of the subject to interpret the wellness scores.

The method can comprise generating a recommendation for the subject. Generating the recommendation can comprise comparing the first wellness score, the second wellness score or the aggregate wellness score to a threshold value to determine if an intervention is necessary. If the wellness score is below the threshold value (e.g., 300), the recommendation can be generated. Otherwise, the method can comprise not generating a recommendation. Generating the recommendation can comprise determining a severity of the subject's wellness. For example, if the first wellness score, the second wellness score or the aggregate wellness score is below a second threshold value (e.g., 100), the method can comprise determining that escalated intervention is needed and recommending emergency medical care. If the wellness score is above the second threshold value, the method can comprise generating a more moderate recommendation, such as recommending a lifestyle change. Generating the recommendation can comprise determining the clinical features having the higher level of contribution to the first wellness score, the second wellness score or the aggregate wellness score. For example, if it is determined that the chronic pain is the biggest contributor to a low aggregate wellness score, the recommendation can be focused on pain management.

### Systems

Provided herein, in some aspects, are computer systems for generating a recommendation. In some embodiments, the systems may comprise: a non-transitory memory; a processor in communication with the non-transitory memory, the processor configured to execute the following operations in order to effectuate any methods as described herein comprising the operations of: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation. In some embodiments, the recommendation is directed to an improvement of the wellness state of the second wellness type. In some embodiments, the operations further comprise: determining that an action based on the recommendation has been taken; determining an improvement of the wellness state of the second wellness type based on the taken action; determining an improvement of a wellness state of the first wellness type based on the improvement of the wellness state of the second wellness type; determining a correlation between or among the taken action, the improvement of the wellness state of the second wellness type, and the improvement of the wellness state of the first wellness type; concluding, based on the correlation, that the wellness state of the first wellness type is caused by the wellness state of the second wellness type, or that the wellness state of the second wellness type is not caused by the wellness state of the first wellness type; suggesting a diagnosis with respect to the wellness state of the second wellness type based on the concluding; and suggesting a change to a treatment with respect to the wellness state of the first wellness type or the wellness state of the second wellness type based on the diagnosing.

Further provided herein, in some aspects, is a non-transitory computer-readable memory storing one or more instructions executable by one or more processors, that when executed by the one or more processors cause the one or more processors to perform processing of any of the methods as described herein comprising: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation. In some embodiments, the recommendation is directed to an improvement of the wellness state of the second wellness type. In some embodiments, the one or more instructions further comprise: determining that an action based on the recommendation has been taken; determining an improvement of the wellness state of the second wellness type based on the taken action; determining an improvement of a wellness state of the first wellness type based on the improvement of the wellness state of the second wellness type; determining a correlation between or among the taken action, the improvement of the wellness state of the second wellness type, and the improvement of the wellness state of the first wellness type; concluding, based on the correlation, that the wellness state of the first wellness type is caused by the wellness state of the second wellness type, or that the wellness state of the second wellness type is not caused by the wellness state of the first wellness type; suggesting a diagnosis with respect to the wellness state of the second wellness type based on the concluding; and suggesting a change to a treatment with respect to the wellness state of the first wellness type or the wellness state of the second wellness type based on the diagnosing.

### Generating a Recommendation

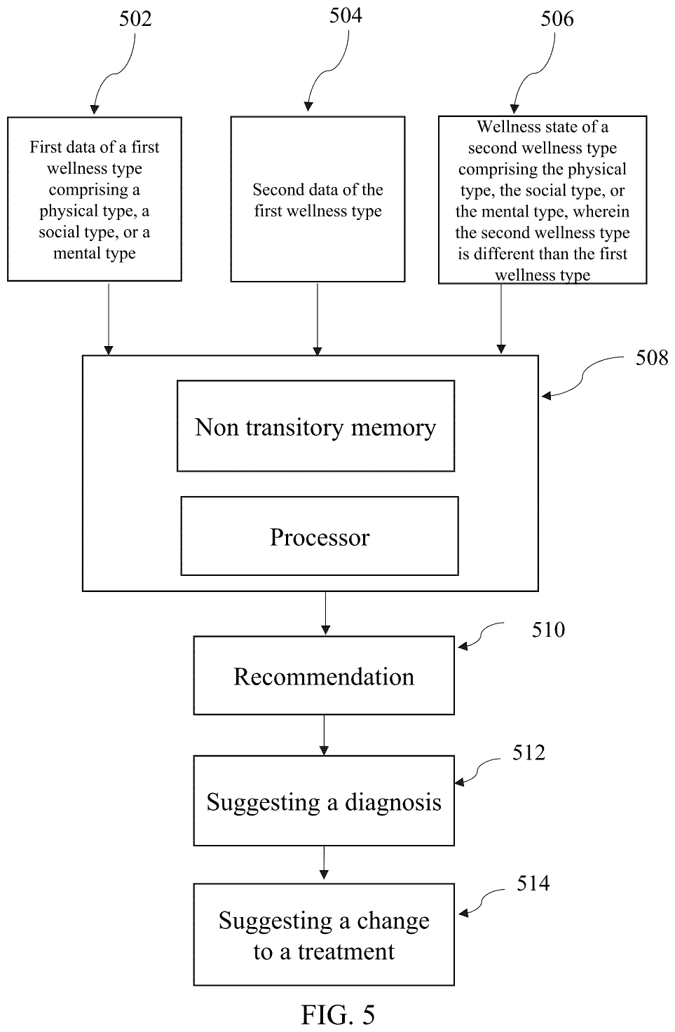

FIG. 5 illustrates an example workflow including a system for generating a recommendation through process operations as described herein.

At operation 502, a first data of a first wellness type is received by system 508. The first wellness type may be a physical type, a social type, or a mental type. The non-transitory memory may include the memory 103 depicted in FIG. 1. The non-transitory memory herein may include a RAM 104, a ROM 105, or a BIOS 106, or a combination thereof. The processor may include a cache 102. The first data of the first wellness type may be received at an application, for example a mobile application or a web-based application. As illustrated in FIG. 2, such data may be received by an application server 230 or an application server 220. The data may be managed on a RDBMS 210, and stored on a database 200.

At operation 504, a second data of the first wellness type of operation 502 is received by system 508. The first and second data of operations 502 and 504, respectively, may be the same type of wellness type. As such, the first and second data be both be a physical type, a social type, or a mental type. The second data of the first wellness type may be received at an application, for example a mobile application or a web-based application. As illustrated in FIG. 2, such data may be received by an application server 230 or an application server 220. The data may be managed on a RDBMS 210, and stored on a database 200.

At operation 506, a wellness state of a second wellness type is received by system 508. The wellness state of the second wellness type may be a physical type, a social type, or a mental type. The second wellness type of operation 506 may be different than the first wellness type of operations 502 and 504. For example, the second wellness type may be a physical type and the first wellness type may be a social type or a mental type. As another example, the second wellness type may be a social type and the first wellness type may be a physical type or a mental type. As yet another example, the second wellness type may be a physical type and the first wellness type may be a social type or a mental type. The wellness state of the second wellness type may be received at an application, for example a mobile application or a web-based application. As illustrated in FIG. 2, the data may be received by an application server 230 or an application server 220. The data may be managed on a RDBMS 210, and stored on a database 200.

System 508 may comprise a computer system, such as computer system 100, an application server 230, or application server 220. System 508 may comprise a non-transitory memory and a processor. The non-transitory memory may store the operations of any of the methods described herein, including: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation. In some embodiments, the recommendation is directed to an improvement of the wellness state of the second wellness type. In some embodiments, the operations further comprise: determining that an action based on the recommendation has been taken; determining an improvement of the wellness state of the second wellness type based on the taken action; determining an improvement of a wellness state of the first wellness type based on the improvement of the wellness state of the second wellness type; determining a correlation between or among the taken action, the improvement of the wellness state of the second wellness type, and the improvement of the wellness state of the first wellness type; concluding, based on the correlation, that the wellness state of the first wellness type is caused by the wellness state of the second wellness type, or that the wellness state of the second wellness type is not caused by the wellness state of the first wellness type; suggesting a diagnosis with respect to the wellness state of the second wellness type based on the concluding; and suggesting a change to a treatment with respect to the wellness state of the first wellness type or the wellness state of the second wellness type based on the diagnosing.

System 508 may comprise a storage as depicted in FIG. 1. The storage may include an operating system 109, an exec 110, a data 111, and one or more API Applications.

At process operation 510, system 508 may generate a recommendation. Process operation 610 may utilize any of the methods as described herein to generate the recommendation, and any of the recommendations described herein may be generated at operation 510. As described in the Recommendations section herein, the generated recommendation may be a preprogrammed recommendation, a user-provided recommendation, or a machine learning generated recommendation (see also Machine Learning Techniques section).

At process operation 512, system 508 may generate a suggested diagnosis. Process operation 512 may utilize any of the methods as described herein to generate the suggested diagnosis, and any of the suggested diagnoses described herein may be generated at operation 512. As described in the Diagnosis section herein, the generated suggested diagnosis may be generated by a machine learning technique described herein (see Machine Learning Techniques section).

At process operation 514, system 508 may generate a suggested change in a treatment. Process operation 514 may utilize any of the methods as described herein to generate such suggested change, and any of the suggested changes described herein may be generated at operation 514. Such suggested change may be generated by a machine learning technique described herein (see Machine Learning Techniques section).

Thus, the processor of system 508, which may be in communication with the non-transitory memory, may be configured to execute one or more instructions in order to effectuate any of the methods described herein, including comprising the operations of: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation. In some embodiments, the recommendation is directed to an improvement of the wellness state of the second wellness type. In some embodiments, the one or more instructions further comprise: determining that an action based on the recommendation has been taken; determining an improvement of the wellness state of the second wellness type based on the taken action; determining an improvement of a wellness state of the first wellness type based on the improvement of the wellness state of the second wellness type; determining a correlation between or among the taken action, the improvement of the wellness state of the second wellness type, and the improvement of the wellness state of the first wellness type; concluding, based on the correlation, that the wellness state of the first wellness type is caused by the wellness state of the second wellness type, or that the wellness state of the second wellness type is not caused by the wellness state of the first wellness type; suggesting a diagnosis with respect to the wellness state of the second wellness type based on the concluding; and suggesting a change to a treatment with respect to the wellness state of the first wellness type or the wellness state of the second wellness type based on the diagnosing.

### Computing Systems

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between or among elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used. A network 130 in FIG. 1 may be comprised of any of the elements of FIG. 2. For example, the network 130 may include one or more web servers 230, application programming interfaces (APIs) 240, one or more application servers 220, a relational database management system (RDBMS) 210, or one or more databases 200, or a combination thereof.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC VIVE®, OCULUS RIFT, SAMSUNG GEAR VR®, MICROSOFT HOLOLENS®, RAZER OSVR®, FOVE VR, Zeiss VR One, AVEGANT GLYPH, Freefly VR, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition, or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FREEBSD®, OPENBSD®, NETBSD®, LINUX®, APPLE® MAC OS X SERVER®, ORACLE® SOLARIS®, WINDOWS SERVER®, AND NOVELL® NETWARE®. Those of skill in the art will recognize that suitable personal computer operating systems include, by way of non-limiting examples, MICROSOFT® WINDOWS®, APPLE® MAC OS X®, UNIX®, and UNIX-like operating systems such as GNU/LINUX®. In some embodiments, the operating system is provided by cloud computing. Those of skill in the art will also recognize that suitable mobile smartphone operating systems include, by way of non-limiting examples, NOKIA® SYMBIAN® OS, APPLE® IOS®, RESEARCH IN MOTION® BLACKBERRY OS®, GOOGLE® ANDROID®, MICROSOFT® WINDOWS PHONE® OS, MICROSOFT® WINDOWS MOBILE® OS, LINUX®, and PALM® WEBOS®. Those of skill in the art will also recognize that suitable media streaming device operating systems include, by way of non-limiting examples, APPLE TV®, ROKU®, BOXEE®, GOOGLE TV®, GOOGLE CHROMECAST®, AMAZON FIRE®, and SAMSUNG® HOMESYNC®. Those of skill in the art will also recognize that suitable video game console operating systems include, by way of non-limiting examples, SONY® PS3®, SONY PS4®, MICROSOFT® XBOX 360®, MICROSOFT XBOX ONE, NINTENDO® WII®, NINTENDO® WII U®, and OUYA®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some embodiments, any of the method operations described herein is stored in a storage, including a non-transitory computer readable storage medium. In some embodiments, data is stored in the non-transitory computer readable storage medium. Data may include physical wellness data, mental wellness data, social wellness data, and any other data. In some embodiments, a wellness state is stored in the non-transitory computer readable storage medium. A wellness state may include a physical wellness state, a mental wellness state, a social wellness state, and other wellness states.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®NET or Ruby on Rails (RoR). In some embodiments, a web application utilizes one or more database systems including, by way of non-limiting examples, relational, non-relational, object oriented, associative, XML, and document-oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ACTIONSCRIPT®, JAVASCRIPT®, or SILVERLIGHT®. In some embodiments, a web application is written to some extent in a server-side coding language such as Active Server Pages (ASP), COLDFUSION®, Perl®, JAVA™, JavaServer Pages (JSP), Hypertext Preprocessor (PHP), PYTHON™, RUBY, TCL, SMALLTALK, WebDNA®, or Groovy. In some embodiments, a web application is written to some extent in a database query language such as Structured Query Language (SQL). In some embodiments, a web application integrates enterprise server products such as IBM® LOTUS DOMINO®. In some embodiments, a web application includes a media player element. In various further embodiments, a media player element utilizes one or more of many suitable multimedia technologies including, by way of non-limiting examples, ADOBE® FLASH®, HTML 5, APPLE® QUICKTIME®, MICROSOFT® SILVERLIGHT®, JAVA™, and UNITY®. In some embodiments, the user may input data into a mobile application. The data may be received from the mobile application and stored in one or more databases. The data may be sent to a server.

Referring to FIG. 2, in a particular embodiment, an application provision system comprises one or more databases 200 accessed by a relational database management system (RDBMS) 210. Suitable RDBMSs include Firebird, My SQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces. Referring to FIG. 1, the network 130 may comprise any of the elements in FIG. 2.

In one embodiment, the display on mobile device may be configured to show a user interface (UI) or a graphical user interface rendered through an application (e.g., via an application programming interface (API) executed on the user device). The GUI may display, for example, a user portal with various features such as document upload, query input field, preview of system identified relevant information, physical health status and score, mental health status and score, social health status and score, overall health status and score, and other information and representations of information described herein. A user may input information into a mobile GUI. A user may include a subject or a third person that is not the subject, such as a healthcare professional, a caretaker, or the like. The user may input health information into the, such as health data, including physical wellness data, mental wellness data, and social wellness data, as described herein. The user may upload documents to the application, including medical documents such as medical records and history. The user may upload additional documents such as vaccination records, disease history, familial lineage, and the like. The user may upload information such as where the subject lives, such as the address, city, state, country, and the like, to the application. The application may be configured to receive data from a device comprising a sensor or a sensor (either of which may be referred to herein as a sensor). For example, the user may connect a device, such as a smart watch or ring to the application, and the user may wear such device. The device may automatically provide data of the subject to the application, such as heart rate, hours slept per night, blood oxygen content, steps walked, and the like. The information inputted by the user may be stored in one or more databases 200. The one or more databases 200 may contain stored instructions, that when executed, causes the application to execute any of the methods described herein, including the following operations: receiving a first data of a first wellness type comprising a physical type, a social type, or a mental type; receiving a second data of the first wellness type; receiving a wellness state of a second wellness type comprising the physical type, the social type, or the mental type, wherein the second wellness type is different than the first wellness type; determining a change in the wellness state of the second wellness type based on a change between the first data of the first wellness type and the second data of the first wellness type; and generating, based on the determining, the recommendation.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In some embodiments, a mobile application receives data relating to a subject. For example, the subject may provide data to the mobile application through a GUI. The application may receive wellness data and wellness state information as described herein from the subject, a user, or a sensor. For example, the subject may directly input information into the application. As another example, a healthcare professional may input information of the subject into the application through a GUI or a web portal. As yet another example, a device or sensor such as a camera, smart watch, ring, or implant (comprising, for example, a heart rate sensor, a blood pressure sensor, a temperature sensor, or other sensors) may push wellness and other data to the application using an API or other application interface of the system on which the application runs. The application may also pull data from such device or sensor using an API or other application interface of the device or sensor.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, Airplay SDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, APPLE® APP STORE®, GOOGLE PLAY®, Chrome Web Store, BLACKBERRY® App World, App Store for Palm devices, App Catalog for webOS, WINDOWS® Marketplace for Mobile, Ovi Store for NOKIA® devices, SAMSUNG® Apps, and NINTENDO® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® Quick Time®. In some embodiments, the toolbar comprises one or more web browser extensions, add-ins, or add-ons. In some embodiments, the toolbar comprises one or more explorer bars, tool bands, or desk band s.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, GOOGLE® ANDROID® browser, RIM BLACKBERRY® Browser, APPLE® SAFARI®, PALM Blazer, PALM® WEBOS® Browser, MOZILLA® FIREFOX® for mobile, MICROSOFT® INTERNET EXPLORER® Mobile, AMAZON® KINDLE® Basic Web, NOKIA® Browser, OPERA SOFTWARE® OPERA® Mobile, and SONY® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of health information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices. In some examples, the databases may store instructions that when executed by a processor causes the processor to perform any of the methods described herein.

### Machine Learning (ML) Techniques

As disclosed herein, in some cases, the systems, the methods, the computer-readable media, and the techniques disclosed herein may implement one or more machine learning techniques to identify changes in data, generate recommendations, identify correlations and causations, provide a suggested diagnosis, and suggest a treatment or change thereto. In some cases, machine learning may generally involve identifying and recognizing patterns in existing data, such as data obtained from a monitoring device or a questionnaire, in order to facilitate making predictions for subsequent data. Data may be any input, intermediate output, previous outputs, or training information, or otherwise any information provided to or by the algorithms. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, unsupervised, reinforcement learning, transduction, “learning to learn,” or other ML techniques.

A machine learning algorithm may use a supervised learning approach. In supervised learning, the algorithm can generate a function or model from training data. The training data can be labeled. The training data may include metadata associated therewith. Each training example of the training data may be a pair consisting of at least an input object and a desired output value. A supervised learning algorithm may require the individual to determine one or more control parameters. These parameters and the weights thereof can be adjusted by optimizing performance on a subset, for example a validation set, of the training data. After parameter adjustment and learning, the performance of the resulting function or model can be measured on a test set that may be separate from the training set. Regression methods can be used in supervised learning approaches.

A machine learning algorithm may use an unsupervised learning approach. In unsupervised learning, the algorithm may generate a function/model to describe hidden structures from unlabeled data (i.e., a classification or categorization that cannot be directly observed or computed). The examples given to the learner may be unlabeled, and there may be no evaluation of the accuracy of the structure that is output by the relevant algorithm. Approaches to unsupervised learning include: clustering, anomaly detection, and neural networks.

A machine learning algorithm may use a semi-supervised learning approach. Semi-supervised learning can combine both labeled and unlabeled data to generate an appropriate function or classifier.

A machine learning algorithm may use a reinforcement learning approach. In reinforcement learning, the algorithm can learn a policy of how to act given an observation of the world. Every action may have some impact in the environment, and the environment can provide feedback that guides the learning algorithm.

A machine learning algorithm may use a transduction approach. Transduction can be similar to supervised learning, but does not explicitly construct a function. Instead, transduction tries to predict new outputs based on training inputs, training outputs, and new inputs.

A machine learning algorithm may use a “learning to learn” approach. In learning to learn, the algorithm can learn its own inductive bias based on previous experience.

A machine learning algorithm may be applied to subject data to generate a prediction model. In some embodiments, a machine learning algorithm or model may be trained periodically. In some embodiments, a machine learning algorithm or model may be trained non-periodically.

ML may comprise one or more of regression analysis, regularization, classification, dimensionality reduction, ensemble learning, meta learning, association rule learning, cluster analysis, anomaly detection, deep learning, or ultra-deep learning. ML may comprise: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, large language models, vision transformers, or generative adversarial networks.

Training the ML model may include, in some cases, selecting one or more untrained data models to train using a training data set. The selected untrained data models may include any type of untrained ML models for supervised, semi-supervised, unsupervised, reinforcement, transduction, “learning to learn,” or other machine learning techniques. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some embodiments, the ML model may be stored in a database (e.g., associated with a server).

The machine learning techniques described herein may be utilized in any of the methods described herein. For example, the machine learning techniques may be used to generate a recommendation. In one example, the application may receive data relating to a subject. The application may analyze such data and compare such data to other subjects' data that is stored in a database. Using a machine learning technique such as k-nearest neighbors, or any other suitable technique, the application provides a recommendation based on other similarly situated subjects stored in the database. In another example, the machine learning techniques may be used to determine a correlation and/or causation. For example, an application may receive information of a subject regarding improvement of a wellness state of a first wellness type and a wellness state of a second wellness type. Machine learning may be used to determine whether there is a correlation between or among an action, an improvement in a wellness state of the first wellness type and an improvement in a wellness state of the second wellness type, and the correlation may be expressed as a correlation coefficient or a percentage. Machine learning may also be used to determine whether an improvement in one wellness state causes an improvement of another wellness state.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

What is claimed is:

1. A method for generating a recommendation, comprising:
receiving a first data of a first wellness type comprising a physical type, a social type, or a mental type;
receiving a second data of the first wellness type;
receiving a wellness state of a second wellness type comprising the physical type, the social type, or the mental type, wherein the second wellness type is different than the first wellness type;
determining a change in the wellness state of the second wellness type based on a change between the first data of the first wellness type and the second data of the first wellness type; and
generating, based on the determining, the recommendation.

2. The method of claim 1, wherein the recommendation is directed to an improvement of the wellness state of the second wellness type.

3. The method of claim 2, further comprising determining that an action based on the recommendation has been taken.

4. The method of claim 3, further comprising determining an improvement of the wellness state of the second wellness type based on the taken action.

5. The method of claim 4, further comprising determining an improvement of a wellness state of the first wellness type based on the improvement of the wellness state of the second wellness type.

6. The method of claim 5, further comprising determining a correlation between or among the taken action, the improvement of the wellness state of the second wellness type, and the improvement of the wellness state of the first wellness type.

7. The method of claim 6, further comprising concluding, based on the correlation, that the improvement to the wellness state of the first wellness type is caused by the improvement to the wellness state of the second wellness type, or that the change to the wellness state of the second wellness type is not caused by the change between the first data and the second data.

8. The method of claim 7, further comprising suggesting a diagnosis with respect to the wellness state of the second wellness type based on the concluding.

9. The method of claim 8, further comprising suggesting a change to a treatment with respect to the wellness state of the first wellness type or the wellness state of the second wellness type based on the diagnosing.

10. A computer system for generating a recommendation, comprising:
a non-transitory memory; and
a processor in communication with the non-transitory memory, the processor configured to execute the following operations in order to effectuate a method comprising the operations of:
receiving a first data of a first wellness type comprising a physical type, a social type, or a mental type;
receiving a second data of the first wellness type;
receiving a wellness state of a second wellness type comprising the physical type, the social type, or the mental type, wherein the second wellness type is different than the first wellness type;
determining a change in the wellness state of the second wellness type based on a change between the first data of the first wellness type and the second data of the first wellness type; and
generating, based on the determining, the recommendation.

11. The system of claim 10, wherein the recommendation is directed to an improvement of the wellness state of the second wellness type.

12. The system of claim 11, wherein the method further comprises determining that an action based on the recommendation has been taken.

13. The system of claim 12, wherein the method further comprises determining an improvement of the wellness state of the second wellness type based on the taken action.

14. The system of claim 13, wherein the method further comprises determining an improvement of a wellness state of the first wellness type based on the improvement of the wellness state of the second wellness type.

15. A method for determining a wellness score for a wellness type of a subject comprising:
obtaining clinical data associated with the subject;
processing the clinical data to extract a plurality of clinical features;
determining, by at least in part on an algorithm, the wellness score based at least in part on the plurality of clinical features and a plurality of weights, wherein each of the weights in the plurality of weights is indicative of a level of a contribution of a corresponding clinical feature to the wellness score.

16. The method of claim 15, wherein the clinical data comprises one or more of biometrics measurements, clinical notes, subject input, or medical codes.

17. The method of claim 15, wherein the clinical data comprises text, and wherein extracting the plurality of clinical features comprises processing the clinical data with a large language model (LLM).

18. The method of claim 17, wherein the LLM is pre-trained, trained, or fine-tuned using clinical notes.

19. The method of claim 15, further comprising determining one or more additional wellness scores for one or more additional wellness types of the subject.

20. The method of claim 19, further comprising aggregating the wellness score and the one or more additional wellness scores to generate an aggregate wellness score.
